SEQUENCE LISTING (24 sequences)

SEQ ID NO: 1 - DNA, 20 bp, Artificial Sequence
  Nestin
tgctagccctgcctgtctac

SEQ ID NO: 2 - DNA, 21 bp, Artificial Sequence
  Nestin
catcattgctgctcctctggg

SEQ ID NO: 3 - DNA, 20 bp, Artificial Sequence
  Hes1
acaccggacaaaccaaagac

SEQ ID NO: 4 - DNA, 20 bp, Artificial Sequence
  Hes1
atgccgggagctatctttct

SEQ ID NO: 5 - DNA, 20 bp, Artificial Sequence
  Fabp7
gctttctgcgcaacctggaa

SEQ ID NO: 6 - DNA, 20 bp, Artificial Sequence
  Fabp7
ttgcctagtggcaaagccca

SEQ ID NO: 7 - DNA, 20 bp, Artificial Sequence
  NeuroD1
agcgagtcatgagtgcccag

SEQ ID NO: 8 - DNA, 21 bp, Artificial Sequence
  NeuroD1
gcacagtggattcgtttcccg

SEQ ID NO: 9 - DNA, 20 bp, Artificial Sequence
  Abca1
tacagtggcggcaacaaacg

SEQ ID NO: 10 - DNA, 20 bp, Artificial Sequence
  Abca1
gggctttagggtccatgcct

SEQ ID NO: 11 - DNA, 20 bp, Artificial Sequence
  Abca2
gtctcggaagattggccgga

SEQ ID NO: 12 - DNA, 20 bp, Artificial Sequence
  Abca2
accaaggagcccaaagcact

SEQ ID NO: 13 - DNA, 20 bp, Artificial Sequence
  Abca3
tgctgcccactactgcaaga

SEQ ID NO: 14 - DNA, 20 bp, Artificial Sequence
  Abca3
cctgaggcagccatggaagt

SEQ ID NO: 15 - DNA, 20 bp, Artificial Sequence
  Abcb1a
ggaggccaacatccaccagt

SEQ ID NO: 16 - DNA, 20 bp, Artificial Sequence
  Abca1a
gtgaggctgtctgacgaggg

SEQ ID NO: 17 - DNA, 20 bp, Artificial Sequence
  Abcb1b
tggcaaagccggagagatcc

SEQ ID NO: 18 - DNA, 20 bp, Artificial Sequence
  Abcb1b
ggtcagtgagccagtgctgt

SEQ ID NO: 19 - DNA, 20 bp, Artificial Sequence
  Abcc1
cccacccttgggtctggttt

SEQ ID NO: 20 - DNA, 20 bp, Artificial Sequence
  Abcc1
actccaggcgcttcagttgt

SEQ ID NO: 21 - DNA, 20 bp, Artificial Sequence
  Abcg2
tcaccttactggcttccggg

SEQ ID NO: 22 - DNA, 20 bp, Artificial Sequence
  Abcg2
cgcagggttgttgtagggct

SEQ ID NO: 23 - DNA, 24 bp, Artificial Sequence
  Actin, beta
accaactgggacgacatggagaag

SEQ ID NO: 24 - DNA, 24 bp, Artificial Sequence
  Actin, beta
tacgaccagaggcatacagggaca

(19) United States
(12) Patent Application Publication
(10) Pub. No.: US 2018/0024136 A1
(43) Pub. Date: Jan. 25, 2018

(54) Development of ABCG2-Sensitive Fluorescent Probe For Isolation of ABCG2low Neural Stem/Progenitor Cells

(71) Applicants: National University of Singapore, Singapore (SG); Agency for Science, Technology and Research, Singapore (SG)

(72) Inventors: Young-Tae CHANG, Singapore (SG); Beomsue KIM, Singapore (SG)

(21) Appl. No.: 15/549,868

(22) PCT Filed: Feb. 11, 2016
(86) PCT No.: PCT/SG2016/050067

§ 371 Date: Aug. 9, 2017

Related U.S. Application Data
(60) Provisional application No. 62/114,936, filed on Feb. 11, 2015.

(51) Int. Cl. G01N 33/58; C07D 405/10; C07D 311/82; C07D 405/12; C07D 413/10

(52) U.S. Cl. CPC G01N 33/582; C07D 405/12; C07D 413/10; C07D 311/82; C07D 405/10

(57) ABSTRACT

The present invention relates to the synthesis and characterization of an Abcg2 targeted fluorescence probe (compound of formula I), as well as live imaging of neural stem/progenitor cells (NSPCs) and isolation of live NSPCs using said probe.

### RELATED APPLICATION

This application claims the benefit of U.S. Provisional Application No. 62/114,936, filed on Feb. 11, 2015. The entire teachings of the above application are incorporated herein by reference.

### BACKGROUND

Neural stem/progenitor cells (NSPCs), a powerful source for the therapy of neurodegenerative disorders and traumatic injuries, are proliferating cells having properties of self-renewal and differentiation into neuron and glia. NSPCs are classified according to their developmental stage and their differentiation capacities, e.g., radial glia (RG) arisen from developing neuroepithelial cells.

Since the current methodology has mainly relied on a limited number of cell surface markers, development of new methods is highly sought after for isolating and applying a minute NSPC population to identify a novel target. Recently, small fluorescent molecules have been employed as a novel tool to visualize and to isolate special cell types ^{1,2}.

ATP binding cassette (ABC) transporters pump out diverse molecules from cells to extracellular spaces in eukaryotes. Side population (SP), defined by fluorescent dye efflux mainly through ABCB1 and/or ABCG2 transporters, has been used to isolate stem cell population from various organs such as hematopoietic and cancer stem cells. However, SP cells from freshly isolated mouse embryonic brain have characteristics of a hematopoietic/endothelial origin, suggesting that NSPCs exist outside of SP³. Hence, analysis of transgenic mice expressing nuclear GFP under Abcg2 promoter also revealed that the majority of NSPCs did not merge with Abcg2 expressing cells⁴. Nonetheless, the study of low Abcg2 expressing types of cells has not been tried because no methods are available to distinguish low levels of Abcg2.

### SUMMARY OF THE INVENTION

The present invention provides a fluorescence probe excluded from a live cell through Abcg2 activity. An isolated population of mouse embryonic brain with strong probe signal showed NSPC properties, enhanced neurosphere forming capacity and neuron/glia differentiation. The population unexpectedly had a high neurogenic potential compared to the conventional CD133^{high} isolated NSPC population from embryonic brain. Thus, the probe of the present invention can be used to isolate a NSPC population having low levels of Abcg2, which retained high neurogenic potential.

In a first aspect, the invention provides a composition represented by structural formula (I):

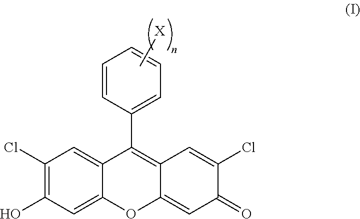

or a salt and/or tautomer thereof, wherein

n is a whole number selected from 1 to 5;

X for each occurrence is independently selected from H, (C₁-C₂₀)alkyl, (C₂-C₂₀)alkenyl, (C₂-C₂₀)alkynyl, (C₁-C₂₀)alkoxy, (C₁-C₂₀)alkylamino, (C₃-C₁₀)cycloalkyl, —C(O)R₁, —S(O)₂R₁, amino, pyridyl, nitrile, nitro or —C(O)N(R₁)(R₂);

R₁ is H, amino, (C₁-C₂₀)alkyl, (C₂-C₂₀)alkenyl, (C₂-C₂₀)alkynyl, (C₁-C₂₀)alkoxy, (C₁-C₂₀)alkylamino or (C₃-C₁₀)cycloalkyl, optionally substituted with one or more groups independently selected from (C₁-C₁₀)alkyl, (C₃-C₁₀)cycloalkyl, halo, (C₆-C₁₂)aryl, (5-12 atom) heteroaryl, (5-12 atom) heterocycle, or —C(O)O(C₁-C₃)alkyl, further optionally substituted with one or more groups selected from halo, (C₆-C₁₂)aryl, (C₁-C₃)alkyl, (C₁-C₃)alkoxy, —OCF₃ or oxo;

R₂ is H, amino, (C₁-C₂₀)alkyl, (C₂-C₂₀)alkenyl, (C₂-C₂₀)alkynyl, (C₁-C₂₀)alkoxy, (C₁-C₂₀)alkylamino or (C₃-C₁₀)cycloalkyl, optionally substituted with one or more groups independently selected from (C₁-C₁₀)alkyl, (C₃-C₁₀)cycloalkyl, halo, (C₆-C₁₂)aryl, (5-12 atom) heteroaryl, (5-12 atom) heterocycle, or —C(O)O(C₁-C₃)alkyl, further optionally substituted with one or more groups selected from halo, (C₆-C₁₂)aryl, (C₁-C₃)alkyl, (C₁-C₃)alkoxy, —OCF₃ or oxo;

or R₁ and R₂ may be taken together to form a ring, wherein the ring is optionally substituted with one or more groups selected from (C₁-C₁₀)alkyl, (C₃-C₁₀)cycloalkyl, halo, (C₆-C₁₂)aryl, (5-12 atom) heteroaryl, (5-12 atom) heterocycle, —C(O)O(C₁-C₃)alkyl, or a 4-5 member polycyclyl fused to the ring, further optionally substituted with one or more groups selected from halo, (C₆-C₁₂)aryl, (C₁-C₃)alkyl, (C₁-C₃)alkoxy, —OCF₃, or oxo;

with the proviso that when the composition of structural formula I is represented by structural formula (II):

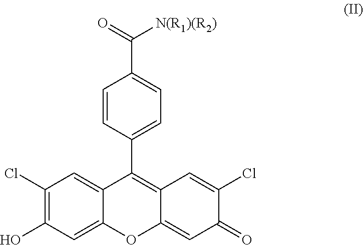

R₁ and R₂ cannot both be n-hexyl.

In an embodiment of the first aspect, X is —C(O)R₁, —S(O)₂R₁ or —C(O)N(R₁)(R₂).

In another embodiment of the first aspect, —C(O)N(R₁)(R₂).

In another embodiment of the first aspect, X is —C(O)N(R₁)(R₂), and R₁ and R₂ are independently (C₅-C₁₂)alkyl.

In another embodiment of the first aspect, X is —C(O)N(R₁)(R₂), and R₁ and R₂ are independently (C₆-C₉)alkyl.

In another embodiment of the first aspect, X is —C(O)N(R₁)(R₂) at para position, and R₁ and R₂ are independently (C₆-C₉)alkyl.

In another embodiment of the first aspect, formula (I) is represented by the structural formula of any of the compounds in Table 2.

In a second aspect, the invention provides a method of visualizing a target cell, the method comprising (a) contacting a population of the target cell with a composition to form an incubation media; (b) incubating the incubation media of step (a) for a period of time sufficient to stain the target cells; and (c) visualizing the stained target cells of step (b) with fluorescence microscopy to visualize the target cell; wherein the composition is represented by structural formula (I):

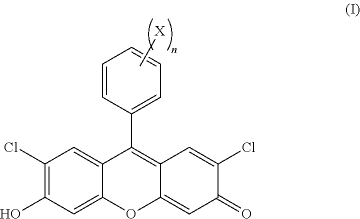

or a salt and/or a tautomer thereof, wherein

n is a whole number selected from 1 to 5;

X for each occurrence is independently selected from H, (C₁-C₂₀)alkyl, (C₂-C₂₀)alkenyl, (C₂-C₂₀)alkynyl, (C₁-C₂₀)alkoxy, (C₁-C₂₀)alkylamino, (C₃-C₁₀)cycloalkyl, —C(O)R₁, —S(O)₂R₁, amino, pyridyl, nitrile, nitro or —C(O)N(R₁)(R₂);

R₁ is H, amino, (C₁-C₂₀)alkyl, (C₂-C₂₀)alkenyl, (C₂-C₂₀)alkynyl, (C₁-C₂₀)alkoxy, (C₁-C₂₀)alkylamino or (C₃-C₁₀)cycloalkyl, optionally substituted with one or more groups independently selected from (C₁-C₁₀)alkyl, (C₃-C₁₀)cycloalkyl, halo, (C₆-C₁₂)aryl, (5-12 atom) heteroaryl, (5-12 atom) heterocycle, or —C(O)O(C₁-C₃)alkyl, further optionally substituted with one or more groups selected from halo, (C₆-C₁₂)aryl, (C₁-C₃)alkyl, (C₁-C₃)alkoxy, —OCF₃ or oxo;

R₂ is H, amino, (C₁-C₂₀)alkyl, (C₂-C₂₀)alkenyl, (C₂-C₂₀)alkynyl, (C₁-C₂₀)alkoxy, (C₁-C₂₀)alkylamino or (C₃-C₁₀)cycloalkyl, optionally substituted with one or more groups independently selected from (C₁-C₁₀)alkyl, (C₃-C₁₀)cycloalkyl, halo, (C₆-C₁₂)aryl, (5-12 atom) heteroaryl, (5-12 atom) heterocycle, or —C(O)O(C₁-C₃)alkyl, further optionally substituted with one or more groups selected from halo, (C₆-C₁₂)aryl, (C₁-C₃)alkyl, (C₁-C₃)alkoxy, —OCF₃ or oxo;

or R₁ and R₂ may be taken together to form a ring, wherein the ring is optionally substituted with one or more groups selected from (C₁-C₁₀)alkyl, (C₃-C₁₀)cycloalkyl, halo, (C₆-C₁₂)aryl, (5-12 atom) heteroaryl, (5-12 atom) heterocycle, —C(O)O(C₁-C₃)alkyl, or a 4-5 member polycyclyl fused to the ring, further optionally substituted with one or more groups selected from halo, (C₆-C₁₂)aryl, (C₁-C₃)alkyl, (C₁-C₃)alkoxy, —OCF₃, or oxo.

In an embodiment of the second aspect, the target cell is a neural stem cell. The neural stem cell can be an ABCG2^{low} neural stem cell.

In another embodiment of the second aspect, X is —C(O)R₁, —S(O)₂R₁ or —C(O)N(R₁)(R₂).

In another embodiment of the second aspect, X is —C(O)N(R₁)(R₂).

In another embodiment of the second aspect, X is —C(O)N(R₁)(R₂), and R₁ and R₂ are independently (C₅-C₁₂)alkyl.

In another embodiment of the second aspect, X is —C(O)N(R₁)(R₂), and R₁ and R₂ are independently (C₆-C₉)alkyl.

In another embodiment of the second aspect, X is —C(O)N(R₁)(R₂) at para position, and R₁ and R₂ are independently (C₆-C₉)alkyl.

In a third aspect, the invention provides a method of isolating a neural stem cell, the method comprising (a) visualizing the neural stem cell by contacting a population of the neural stem cells with a composition to form an incubation media, incubating the incubation media for a period of time sufficient to stain the neural stem cells, and visualizing the stained neural stem cells with fluorescence microscopy to visualize the neural stem cell; (b) exciting the neural stem cells by exposing the incubation media to light of a wavelength of about 488 nm to about 561 nm; and (c) separating the excited neural stem cells from the incubation media by fluorescence activated cell sorting using a bandpass filter configured to detect light emitted at about 529±28 nm; wherein the composition is represented by structural formula (I):

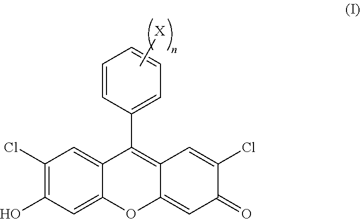

or a salt and/or tautomer thereof, wherein

n is a whole number selected from 1 to 5;

X for each occurrence is independently selected from H, (C₁-C₂₀)alkyl, (C₂-C₂₀)alkenyl, (C₂-C₂₀)alkynyl, (C₁-C₂₀)alkoxy, (C₁-C₂₀)alkylamino, (C₃-C₁₀)cycloalkyl, —C(O)R₁, —S(O)₂R₁, amino, pyridyl, nitrile, nitro or —C(O)N(R₁)(R₂);

R₁ is H, amino, (C₁-C₂₀)alkyl, (C₂-C₂₀)alkenyl, (C₂-C₂₀)alkynyl, (C₁-C₂₀)alkoxy, (C₁-C₂₀)alkylamino or (C₃-C₁₀)cycloalkyl, optionally substituted with one or more groups independently selected from (C₁-C₁₀)alkyl, (C₃-C₁₀)cycloalkyl, halo, (C₆-C₁₂)aryl, (5-12 atom) heteroaryl, (5-12 atom) heterocycle, or —C(O)O(C₁-C₃)alkyl, further optionally substituted with one or more groups selected from halo, (C₆-C₁₂)aryl, (C₁-C₃)alkyl, (C₁-C₃)alkoxy, —OCF₃ or oxo;

R₂ is H, amino, (C₁-C₂₀)alkyl, (C₂-C₂₀)alkenyl, (C₂-C₂₀)alkynyl, (C₁-C₂₀)alkoxy, (C₁-C₂₀)alkylamino or (C₃-C₁₀)cycloalkyl, optionally substituted with one or more groups independently selected from (C₁-C₁₀)alkyl, (C₃-C₁₀)cycloalkyl, halo, (C₆-C₁₂)aryl, (5-12 atom) heteroaryl, (5-12 atom) heterocycle, —C(O)O(C₁-C₃)alkyl, further optionally substituted with one or more groups selected from halo, (C₆-C₁₂)aryl, (C₁-C₃)alkyl, (C₁-C₃)alkoxy, —OCF₃ or oxo;

or R₁ and R₂ may be taken together to form a ring, wherein the ring is optionally substituted with one or more groups selected from (C₁-C₁₀)alkyl, (C₃-C₁₀)cycloalkyl, halo, (C₆-C₁₂)aryl, (5-12 atom) heteroaryl, (5-12 atom) heterocycle, —C(O)O(C₁-C₃)alkyl, or a 4-5 member polycyclyl fused to the ring, further optionally substituted with one or more groups selected from halo, (C₆-C₁₂)aryl, (C₁-C₃)alkyl, (C₁-C₃)alkoxy, —OCF₃, or oxo.

In an embodiment of the third aspect, X is —C(O)R₁, —S(O)₂R₁ or —C(O)N(R₁)(R₂).

In another embodiment of the third aspect, X is —C(O)N(R₁)(R₂).

In another embodiment of the third aspect, X is —C(O)N(R₁)(R₂), and R₁ and R₂ are independently (C₅-C₁₂)alkyl.

In another embodiment of the third aspect, X is —C(O)N(R₁)(R₂), and R₁ and R₂ are independently (C₆-C₉)alkyl.

In another embodiment of the third aspect, X is —C(O)N(R₁)(R₂) at para position, and R₁ and R₂ are independently (C₆-C₉)alkyl.

### DESCRIPTION OF THE DRAWINGS

FIGS. 1A-1B show NMR spectra of compound 1: (A)¹H NMR spectra of compound 1 in DMSO-d⁶, and (B)¹³C NMR spectra of compound 1 in DMSO-d⁶.

FIGS. 2A-2B show NMR spectra of compound 2: (A)¹H NMR spectra of compound 2 in DMSO-d⁶, and (B)¹³C NMR spectra of compound 2 in DMSO-d⁶.

FIGS. 3A-3B show NMR spectra of CDg13: (A)¹H NMR spectra of compound CDg13 in DMSO-d⁶, and (B)¹³C NMR spectra of CDg13 in DMSO-d⁶.

FIGS. 4A-4C show that CDg13 stains ER of NSPC: (A) chemical structure of CDg13, (B) image of live MEF, mouse ESC, NS5 and differentiated NS5 (D-NS5) taken after staining followed by washing briefly (live cells were stained with CDg13 (1 μM) and Hoechst 33342 (2 μM) for 1 hour) (bar: 50 μm), and (C) confocal images of live NS-5 cells stained with CDg13 and a subcellular organelle marker (subcellular organelles were stained with ER, Golgi, lysosome and mitochondria tracker) (bar: 10 μm).

FIGS. 5A-5C show that radial glia cells are enriched in CDg13^{bright} population: (A) live CDg13^{bright} or CD133^{high} cell population (about 5% of total single cells) were sorted from E14.5 mice embryonic cortical brain, (B) phase contrast image of unsorted or sorted neurospheres cultured for 6 days (left) and the number of neurospheres having >50 μm diameter from 10,000 cells/well (N=3) (right) (bar: 200 μm), and (C) the number of neurospheres sub-cultured with 1,000 cells/well—serial passaging of neurospheres were repeated and counted until passage 4 (N=3) (data are mean±SEM. *, p<0.05; **, p<0.01; unpaired two-tailed Student's T-test were used to calculate statistical significance).

FIGS. 6A-6H show neuronal differentiation of CDg13^{bright} neurospheres: (A) differentiated neurospheres were classified as multi- (grey to black) or uni-potent (white) neurospheres; the number of neurospheres were counted and represented as percentage; number of neuron clumps in each multi-potent neurospheres were counted and classified as indicated; total 150 differentiated neurospheres in each group from three independent experiments were analyzed, (B) representative images of multi- (Tuj1⁺GFAP⁺ sphere), or uni-potent (GFAP⁺ sphere) neurosphere (bar: 200 μm), (C) representative image of neurite outgrowth in an edge of a multi-potent neurosphere (bar: 100 μm), (D) average length of neurite outgrowth of a cell from neurospheres were quantified (bar: 100 μm), (E) and (F) protein expression of Tuj1 and β-actin of differentiated neurospheres derived from unsorted, CDg13-, and CD133-sorted cells, actin was used as loading control, representative image of Western blot (E), and (G) and (H) gene expression profile of CDg13^{bright} and CD133high cells isolated from E14.5 mouse embryonic brain, indicated markers of radial glial cells (G) and neurogenic progenitor cells (H) were analyzed by qRT-PCR using total RNA, quantified values of three independent experiments (F) (*, p<0.05;**, p<0.01 compare to the value of unsorted neurospheres (F) or cells (G and H), values are mean±SEM, at least three independent samples were used to analyze each experimental group, unpaired two-tailed Student's T-test were performed to calculate statistical significance).

FIGS. 7A-7C show CDg13 stains depending on Abcg2 activity: (A) CDg13 (1 μM) were stained for 1 hour in live (A-1) or dead cells treated with 4% PFA (A-2) (representative image of three independent experiments, and (B) and (C) NS5 cells were treated STF31 (2 μM), ionomycin (0.1 μM) or Ko143 (0.5 μM) with treatment of CDg13 and Hoechst33342 under normal media (control had same amount of DMSO (0.2%) with other drugs treated group), staining intensity is shown by epifluorescence microscopy (B) or flow cytometry (C) (bar: 50 μm).

FIGS. 8A-8D show that Abcg2 mediates the staining of CDg13: (A) and (B) differentiated NS5 cells were treated with ABC inhibitors during the staining of CDg13 and Hoechst33342, the amount of each inhibitor used: 1 and 5 μM for elacridar (Ela) and Ko143; 10 and 50 μM for verapamil (Vera) and MK571; 100 and 500 μM for probenecid (Pro), representative images (A) are from low concentration of each drug, stained cells were washed with N2 supplement- and serum-free growth medium while observation to prevent further wash-out of CDg13 (control has same amount of DMSO (0.2%) with other drugs treated group), the average intensity from three independent experiments was calculated using flow cytometry (B), and (C) and (D) live cells from P2-4 neurospheres were sorted with CDg13^{bright} (5%) and CDg13^{dim} (10-20% of total) population using FACS, the mRNA expression of ABC transporters of CDg13^{bright} and CDg13^{dim} population were analyzed by qRT-PCR (data were normalized by β-actin expression and represented as relative fold compared to CDg13^{dim} population (N=3).

FIGS. 9A-9D show gene expression of ABC transporters: (A) undifferentiated (NS5) and 3-days differentiated NS5 (D-NS5), (B) the mRNA expression of the indicated ABC transporters were analyzed by qRT-PCR using total RNA from control and Abcg2-targeted siRNA transfected D-NS5 cells (N=3), and (C) and (D) negative (−) control siRNA (siCon) and Abcg2-targeted siRNA (siAbcg2) treated D-NS5 cells were stained with CDg13 for 1 hour, representative cells image (C) and the intensity staining of CDg13 (D) are shown (N=3) (data were normalized by β-actin expression and represented as relative fold, all values are means±SEM, *, p<0.05; **, p<0.01, unpaired two-tailed Student's T-test was used to calculate statistical significance)(Bar in (C): 100 μm).

FIGS. 10A-10D show CDg13 is a substrate of human ABCG2: (A) and (B) human KB3-1 cells (WT) and hABCG2-overexpressed KB3-1 cells (ABCG2) were stained with 1 μM of CDg13, Hoechst 33342 and Rhodamine123 for 1 hour, representative image of stained cells with each probe are shown (A), the intensity of intracellular fluorescence was measured by flow cytometry (N=3)(B), (C) serial concentration of an ABCG2 inhibitor (Ko143) (C) or ABCB1 inhibitor (Verapamil) (D) were pretreated to ABCG2 overexpressing cells (C) or HCT-15 (D) (fluorescence intensity of three fluorescent probes were measured by flow cytometry after 1 hour staining (N=3), all values are means±SEM, **, p<0.01).

FIGS. 11A-11B show CDg13 has high sensitivity to ABCG2: (A) and (B) pre-treatment of RPMI-8226 cells with or without Ko143 were conducted for 30 minutes prior to staining with 1 μM of CDg13, pheophorbide A (PhA) or CDr3, fold change of fluorescence intensity from 3-6 independent experiments (A) (values are means±SEM, *, p<0.05; **, p<0.01), and representative fluorescence histogram of CDg13 and PhA (B) (unstained, 0, 100, 1,000 and 5,000 nM of Ko143 treated group is presented).

FIGS. 12A-12B shows CDg13 has low cytotoxicity: (A) KB3-1 cells were cultured with the indicated amount of Hoechst33342 and CDg13 for 48 hours, MTS assay was conducted to measure their viability (N=3) (values are means±SEM, unpaired two-tailed Student's T-test), and (B) the diameter of 200 neurospheres (P3) cultured for 7 DIV with or without 1 μM of CDg13 are presented as dot plot, red line indicates the median values of size of neurospheres (data is representative of the three independent experiments, n.s.=no significance).

FIG. 13 shows normalised absorption and emission spectra of CDg13 in ethanol (emission: about 550 nm to about 588 nm/normalized intensity 0.5-1.0).

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

### DETAILED DESCRIPTION OF THE INVENTION

A description of example embodiments of the invention follows.

The present invention provides ABCG2-targeted NSPC fluorescent probes, e.g., CDg13 and CF-DC8, selected from a diversity-oriented fluorescence library approach (DOFLA). NSPCs can be easily isolated and purified by using the fluorescent probes of the present invention, e.g., CDg13 and CF-DC8, based on their lowest Abcg2 activity.

A CF library was synthesized by using 4-(2,7-dichloro-3,6-dihydroxy-9H-xanthen-9-yl)benzoic acid, an amine building block and HBTU with DIEA (see Scheme 1, Table 1, Table 2). The spectroscopic properties of the compounds in CF-Library were summarized in Table 3.

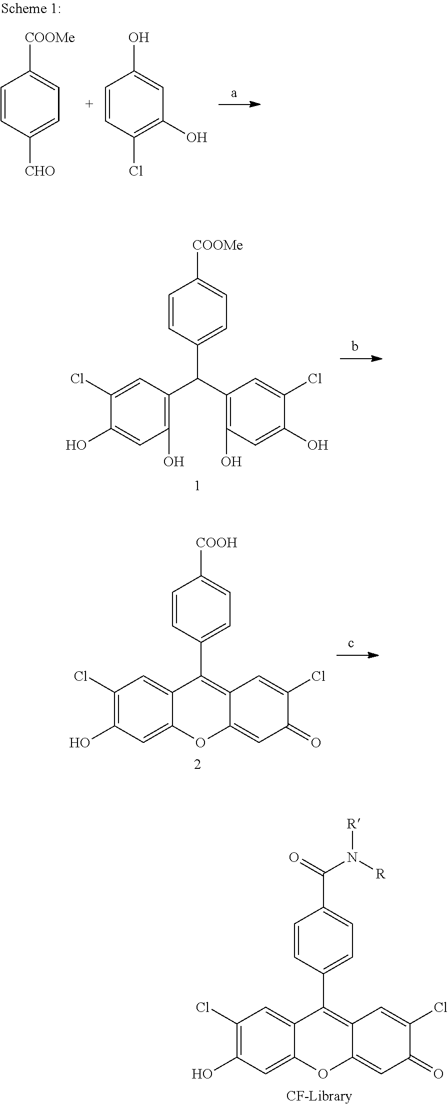

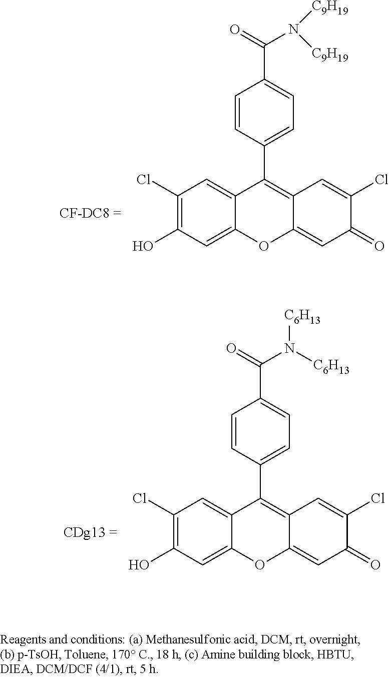

TABLE 1
CF-Plate Map with amine codes

| CF | 1 | 2 | 3 | 4 | 5 | 6 | 7 | 8 | 9 | 10 | 11 | 12 |
|---|---|---|---|---|---|---|---|---|---|---|---|---|
| A | | 28 | 32 | 55 | 66 | 77 | 80 | 115 | 164 | 166 | 171 | |
| | | CF A02 | CF A03 | CF A04 | CF A05 | CF A06 | CF A07 | CF A08 | CF A09 | CF A10 | CF A11 | |
| B | | 175 | 177 | 188 | 193 | 195 | 215 | 222 | 230 | 266 | 277 | |
| | | CF B02 | CF B03 | CF B04 | CF B05 | CF B06 | CF B07 | CF B08 | CF B09 | CF B10 | CF B11 | |
| C | | 349 | 384 | 387 | 395 | 428 | 429 | 442 | 443 | 478 | 582 | |
| | | CF C02 | CF C03 | CF C04 | CF C05 | CF C06 | CF C07 | CF C08 | CF C09 | CF C10 | CF C11 | |
| D | | 11 | 20 | 33 | 34 | 65 | 74 | 78 | 95 | 100 | 131 | |
| | | CF D02 | CF D03 | CF D04 | CF D05 | CF D06 | CF D07 | CF D08 | CF D09 | CF D10 | CF D11 | |
| E | | 135 | 157 | 554 | 180 | 182 | 184 | 185 | 199 | 201 | 206 | |
| | | CF E02 | CF E03 | CF E04 | CF E05 | CF E06 | CF E07 | CF E08 | CF E09 | CF E10 | CF E11 | |
| F | | 219 | 223 | 227 | 232 | 269 | 271 | 273 | 274 | 279 | 292 | |
| | | CF F02 | CF F03 | CF F04 | CF F05 | CF F06 | CF F07 | CF F08 | CF F09 | CF F10 | DCF F11 | |
| G | | 307 | 311 | 335 | 343 | 357 | 373 | 377 | 382 | 419 | 422 | |
| | | CF G02 | CF G03 | CF G04 | CF G05 | CF G06 | CF G07 | CF G08 | CF G09 | CF G10 | DCF G11 | |
| H | | 424 | 426 | 439 | 441 | 462 | 477 | 480 | 599 | 602 | 657 | |
| | | CF H02 | CF H03 | CF H04 | CF H05 | CF H06 | CF H07 | CF H08 | CF H09 | CF H10 | CF H11 | |

TABLE 2
Structures of CF Library Compounds

| Code | Structure |
|---|---|
| 11 | 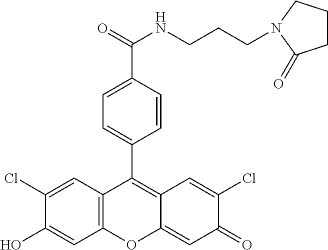 [SMILES: O=C(NCCCN1CCCC1=O)c1ccc(-c2c3cc(Cl)c(=O)cc-3oc3cc(O)c(Cl)cc23)cc1] |
| 20 | 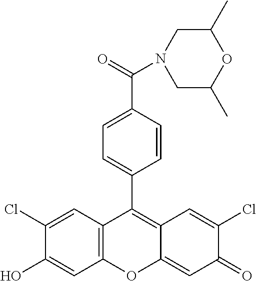 [SMILES: CC1CN(C(=O)c2ccc(-c3c4cc(Cl)c(=O)cc-4oc4cc(O)c(Cl)cc34)cc2)CC(C)O1] |
| 28 | 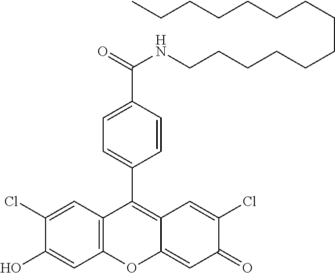 [SMILES: CCCCCCCCCCCCCCNC(=O)c1ccc(-c2c3cc(Cl)c(=O)cc-3oc3cc(O)c(Cl)cc23)cc1] |
| 32 | 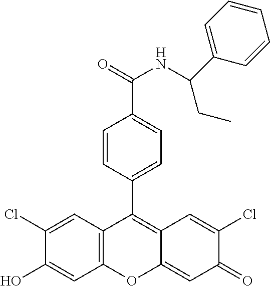 [SMILES: CCC(NC(=O)c1ccc(-c2c3cc(Cl)c(=O)cc-3oc3cc(O)c(Cl)cc23)cc1)c1ccccc1] |
| 33 | 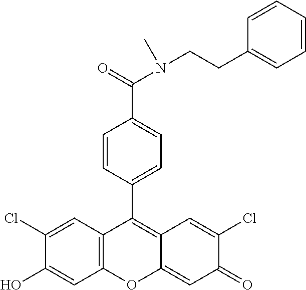 [SMILES: CN(CCc1ccccc1)C(=O)c1ccc(-c2c3cc(Cl)c(=O)cc-3oc3cc(O)c(Cl)cc23)cc1] |
| 34 | 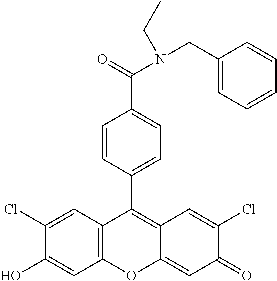 [SMILES: CCN(Cc1ccccc1)C(=O)c1ccc(-c2c3cc(Cl)c(=O)cc-3oc3cc(O)c(Cl)cc23)cc1] |
| 55 | 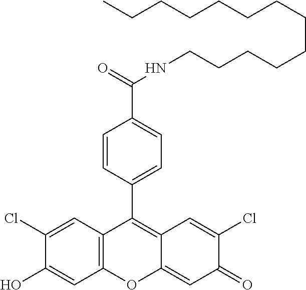 [SMILES: CCCCCCCCCCCCCNC(=O)c1ccc(-c2c3cc(Cl)c(=O)cc-3oc3cc(O)c(Cl)cc23)cc1] |
| 65 | 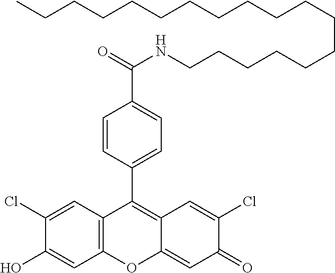 [SMILES: CCCCCCCCCCCCCCCCCCNC(=O)c1ccc(-c2c3cc(Cl)c(=O)cc-3oc3cc(O)c(Cl)cc23)cc1] |
| 66 | 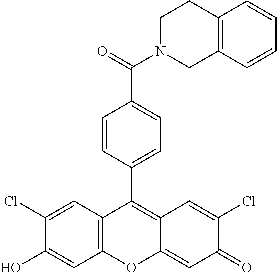 [SMILES: O=C(c1ccc(-c2c3cc(Cl)c(=O)cc-3oc3cc(O)c(Cl)cc23)cc1)N1CCc2ccccc2C1] |
| 74 | 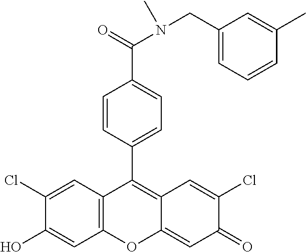 [SMILES: Cc1cccc(CN(C)C(=O)c2ccc(-c3c4cc(Cl)c(=O)cc-4oc4cc(O)c(Cl)cc34)cc2)c1] |
| 77 | 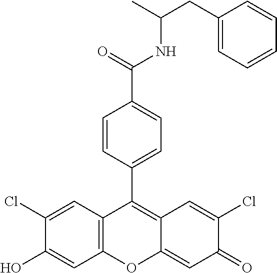 [SMILES: CC(Cc1ccccc1)NC(=O)c1ccc(-c2c3cc(Cl)c(=O)cc-3oc3cc(O)c(Cl)cc23)cc1] |
| 78 | 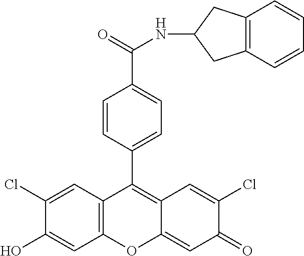 [SMILES: O=C(NC1Cc2ccccc2C1)c1ccc(-c2c3cc(Cl)c(=O)cc-3oc3cc(O)c(Cl)cc23)cc1] |
| 80 | 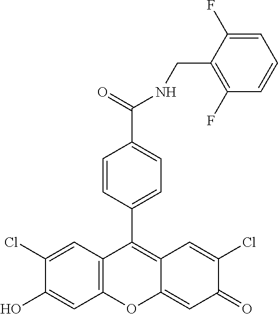 [SMILES: O=C(NCc1c(F)cccc1F)c1ccc(-c2c3cc(Cl)c(=O)cc-3oc3cc(O)c(Cl)cc23)cc1] |
| 95 | 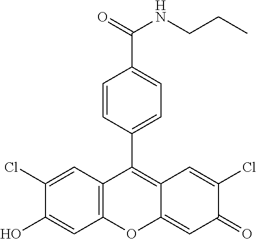 [SMILES: CCCNC(=O)c1ccc(-c2c3cc(Cl)c(=O)cc-3oc3cc(O)c(Cl)cc23)cc1] |
| 100 | 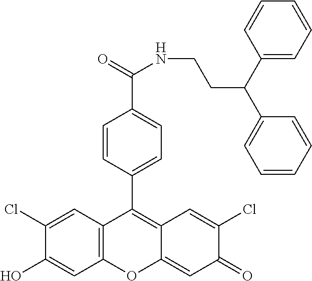 [SMILES: O=C(NCCC(c1ccccc1)c1ccccc1)c1ccc(-c2c3cc(Cl)c(=O)cc-3oc3cc(O)c(Cl)cc23)cc1] |
| 115 | 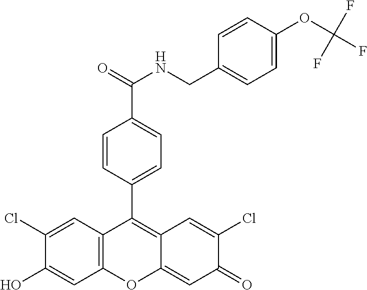 [SMILES: O=C(NCc1ccc(OC(F)(F)F)cc1)c1ccc(-c2c3cc(Cl)c(=O)cc-3oc3cc(O)c(Cl)cc23)cc1] |
| 131 | 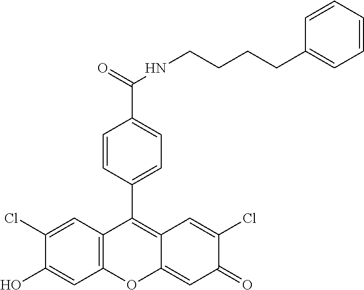 [SMILES: O=C(NCCCCc1ccccc1)c1ccc(-c2c3cc(Cl)c(=O)cc-3oc3cc(O)c(Cl)cc23)cc1] |
| 135 | 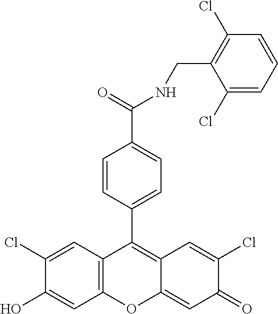 [SMILES: O=C(NCc1c(Cl)cccc1Cl)c1ccc(-c2c3cc(Cl)c(=O)cc-3oc3cc(O)c(Cl)cc23)cc1] |
| 157 | 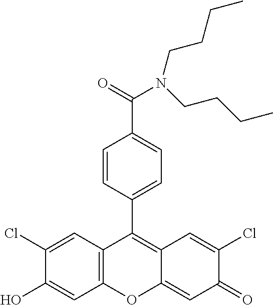 [SMILES: CCCCN(CCCC)C(=O)c1ccc(-c2c3cc(Cl)c(=O)cc-3oc3cc(O)c(Cl)cc23)cc1] |
| 164 | 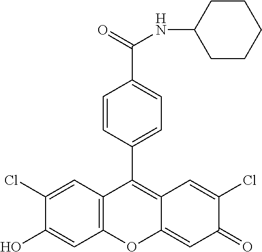 [SMILES: O=C(NC1CCCCC1)c1ccc(-c2c3cc(Cl)c(=O)cc-3oc3cc(O)c(Cl)cc23)cc1] |
| 166 | 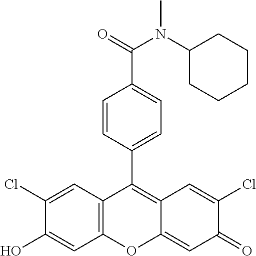 [SMILES: CN(C(=O)c1ccc(-c2c3cc(Cl)c(=O)cc-3oc3cc(O)c(Cl)cc23)cc1)C1CCCCC1] |
| 167 | 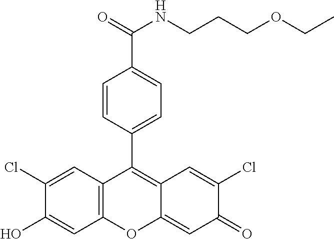 [SMILES: CCOCCCNC(=O)c1ccc(-c2c3cc(Cl)c(=O)cc-3oc3cc(O)c(Cl)cc23)cc1] |
| 171 | 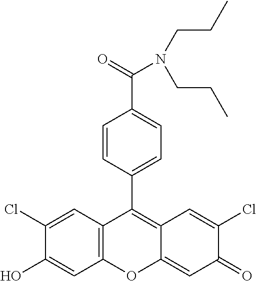 [SMILES: CCCN(CCC)C(=O)c1ccc(-c2c3cc(Cl)c(=O)cc-3oc3cc(O)c(Cl)cc23)cc1] |
| 175 | 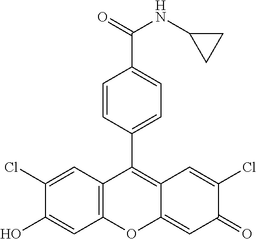 [SMILES: O=C(NC1CC1)c1ccc(-c2c3cc(Cl)c(=O)cc-3oc3cc(O)c(Cl)cc23)cc1] |
| 177 | 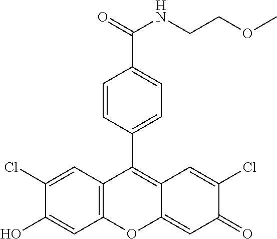 [SMILES: COCCNC(=O)c1ccc(-c2c3cc(Cl)c(=O)cc-3oc3cc(O)c(Cl)cc23)cc1] |
| 180 | 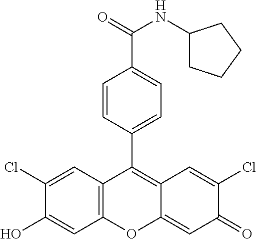 [SMILES: O=C(NC1CCCC1)c1ccc(-c2c3cc(Cl)c(=O)cc-3oc3cc(O)c(Cl)cc23)cc1] |
| 182 | 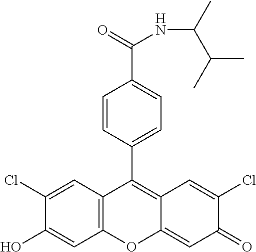 [SMILES: CC(C)C(C)NC(=O)c1ccc(-c2c3cc(Cl)c(=O)cc-3oc3cc(O)c(Cl)cc23)cc1] |
| 184 | 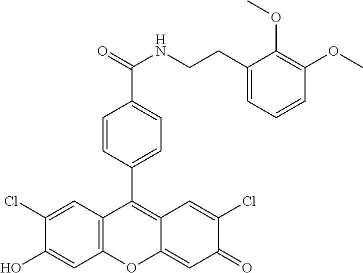 [SMILES: COc1cccc(CCNC(=O)c2ccc(-c3c4cc(Cl)c(=O)cc-4oc4cc(O)c(Cl)cc34)cc2)c1OC] |
| 185 | 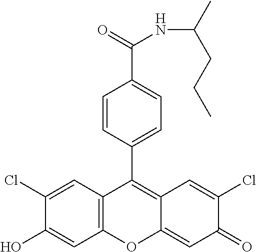 [SMILES: CCCC(C)NC(=O)c1ccc(-c2c3cc(Cl)c(=O)cc-3oc3cc(O)c(Cl)cc23)cc1] |
| 188 | 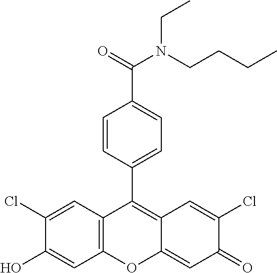 [SMILES: CCCCN(CC)C(=O)c1ccc(-c2c3cc(Cl)c(=O)cc-3oc3cc(O)c(Cl)cc23)cc1] |
| 193 | 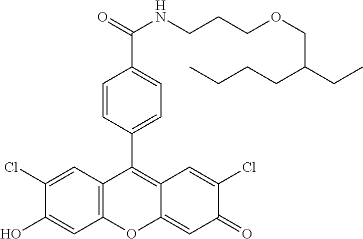 [SMILES: CCCCC(CC)COCCCNC(=O)c1ccc(-c2c3cc(Cl)c(=O)cc-3oc3cc(O)c(Cl)cc23)cc1] |
| 195 | 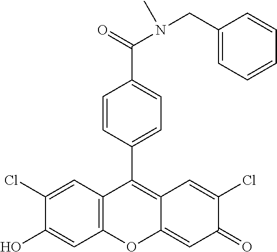 [SMILES: CN(Cc1ccccc1)C(=O)c1ccc(-c2c3cc(Cl)c(=O)cc-3oc3cc(O)c(Cl)cc23)cc1] |
| 199 | 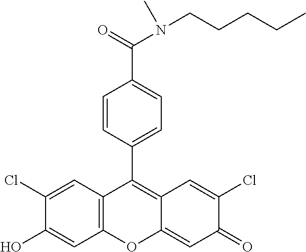 [SMILES: CCCCCN(C)C(=O)c1ccc(-c2c3cc(Cl)c(=O)cc-3oc3cc(O)c(Cl)cc23)cc1] |
| 201 | 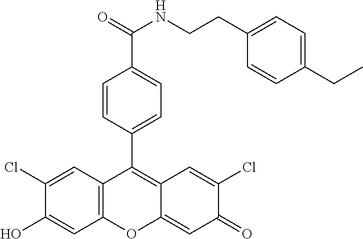 [SMILES: CCc1ccc(CCNC(=O)c2ccc(-c3c4cc(Cl)c(=O)cc-4oc4cc(O)c(Cl)cc34)cc2)cc1] |
| 206 | 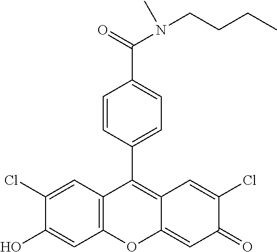 [SMILES: CCCCN(C)C(=O)c1ccc(-c2c3cc(Cl)c(=O)cc-3oc3cc(O)c(Cl)cc23)cc1] |
| 215 | 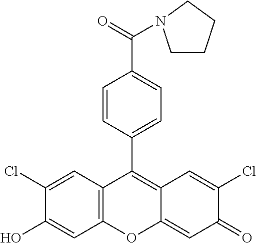 [SMILES: O=C(c1ccc(-c2c3cc(Cl)c(=O)cc-3oc3cc(O)c(Cl)cc23)cc1)N1CCCC1] |
| 219 | 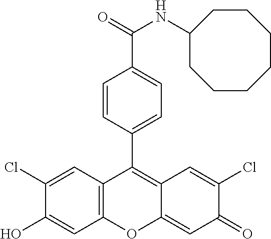 [SMILES: O=C(NC1CCCCCCC1)c1ccc(-c2c3cc(Cl)c(=O)cc-3oc3cc(O)c(Cl)cc23)cc1] |
| 222 | 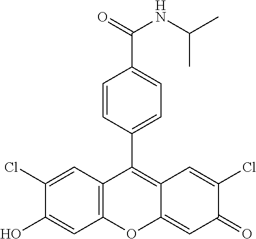 [SMILES: CC(C)NC(=O)c1ccc(-c2c3cc(Cl)c(=O)cc-3oc3cc(O)c(Cl)cc23)cc1] |
| 223 | 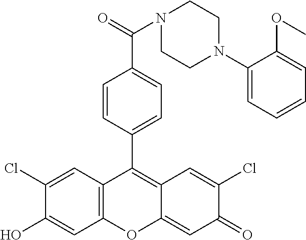 [SMILES: COc1ccccc1N1CCN(C(=O)c2ccc(-c3c4cc(Cl)c(=O)cc-4oc4cc(O)c(Cl)cc34)cc2)CC1] |
| 227 | 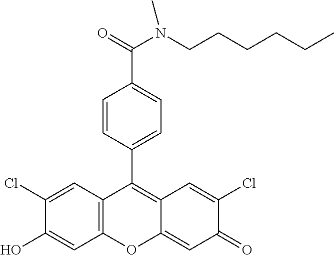 [SMILES: CCCCCCN(C)C(=O)c1ccc(-c2c3cc(Cl)c(=O)cc-3oc3cc(O)c(Cl)cc23)cc1] |
| 230 | 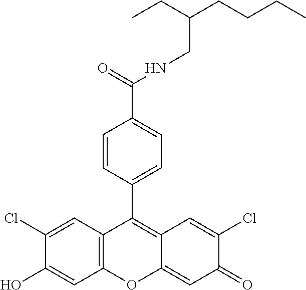 [SMILES: CCCCC(CC)CNC(=O)c1ccc(-c2c3cc(Cl)c(=O)cc-3oc3cc(O)c(Cl)cc23)cc1] |
| 232 | 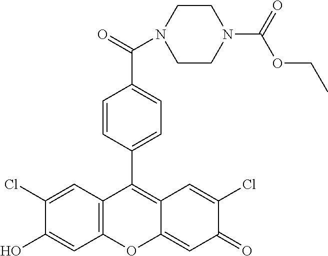 [SMILES: CCOC(=O)N1CCN(C(=O)c2ccc(-c3c4cc(Cl)c(=O)cc-4oc4cc(O)c(Cl)cc34)cc2)CC1] |
| 266 | 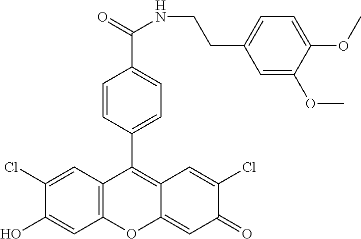 [SMILES: COc1ccc(CCNC(=O)c2ccc(-c3c4cc(Cl)c(=O)cc-4oc4cc(O)c(Cl)cc34)cc2)cc1OC] |
| 269 | 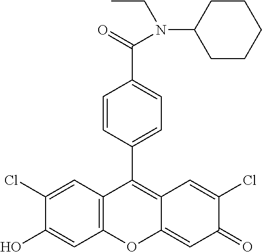 [SMILES: CCN(C(=O)c1ccc(-c2c3cc(Cl)c(=O)cc-3oc3cc(O)c(Cl)cc23)cc1)C1CCCCC1] |
| 271 | 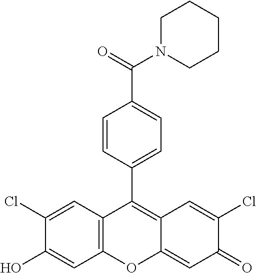 [SMILES: O=C(c1ccc(-c2c3cc(Cl)c(=O)cc-3oc3cc(O)c(Cl)cc23)cc1)N1CCCCC1] |
| 273 | 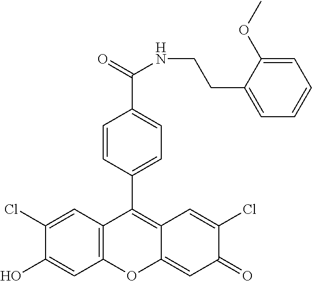 [SMILES: COc1ccccc1CCNC(=O)c1ccc(-c2c3cc(Cl)c(=O)cc-3oc3cc(O)c(Cl)cc23)cc1] |
| 274 | 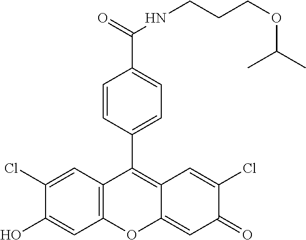 [SMILES: CC(C)OCCCNC(=O)c1ccc(-c2c3cc(Cl)c(=O)cc-3oc3cc(O)c(Cl)cc23)cc1] |
| 277 | 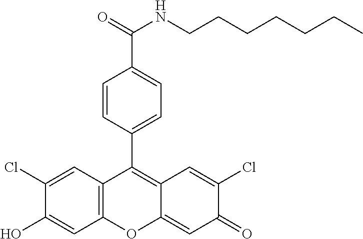 [SMILES: CCCCCCCNC(=O)c1ccc(-c2c3cc(Cl)c(=O)cc-3oc3cc(O)c(Cl)cc23)cc1] |
| 279 | 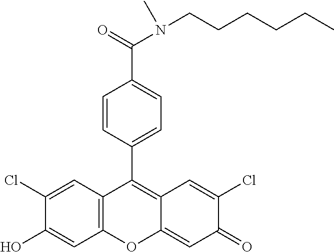 [SMILES: CCCCCCN(C)C(=O)c1ccc(-c2c3cc(Cl)c(=O)cc-3oc3cc(O)c(Cl)cc23)cc1] |
| 292 | 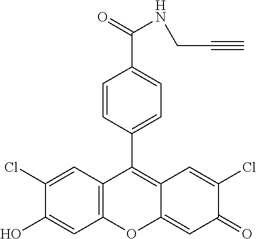 [SMILES: C#CCNC(=O)c1ccc(-c2c3cc(Cl)c(=O)cc-3oc3cc(O)c(Cl)cc23)cc1] |
| 307 | 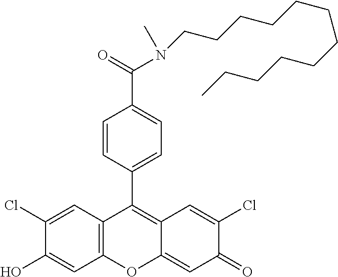 [SMILES: CCCCCCCCCCCCN(C)C(=O)c1ccc(-c2c3cc(Cl)c(=O)cc-3oc3cc(O)c(Cl)cc23)cc1] |
| 311 | 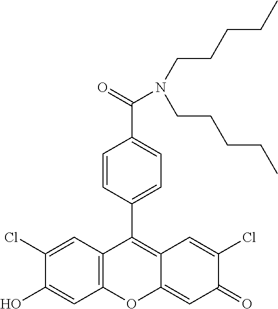 [SMILES: CCCCCN(CCCCC)C(=O)c1ccc(-c2c3cc(Cl)c(=O)cc-3oc3cc(O)c(Cl)cc23)cc1] |
| 335 | 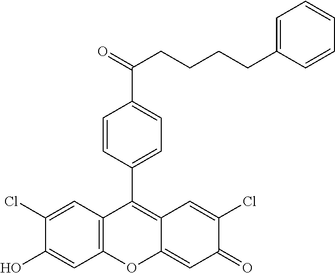 [SMILES: O=C(CCCCc1ccccc1)c1ccc(-c2c3cc(Cl)c(=O)cc-3oc3cc(O)c(Cl)cc23)cc1] |
| 343 | 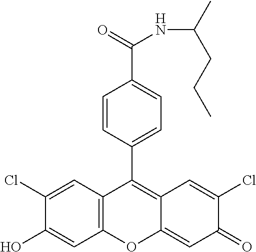 [SMILES: CCCC(C)NC(=O)c1ccc(-c2c3cc(Cl)c(=O)cc-3oc3cc(O)c(Cl)cc23)cc1] |
| 349 | 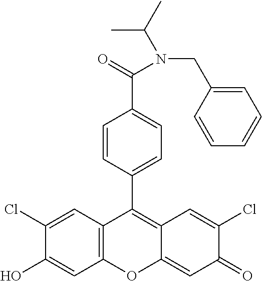 [SMILES: CC(C)N(Cc1ccccc1)C(=O)c1ccc(-c2c3cc(Cl)c(=O)cc-3oc3cc(O)c(Cl)cc23)cc1] |
| 357 | 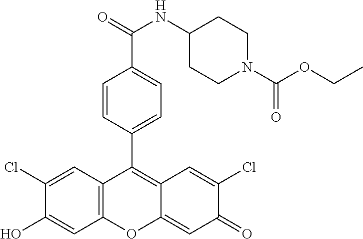 [SMILES: CCOC(=O)N1CCC(NC(=O)c2ccc(-c3c4cc(Cl)c(=O)cc-4oc4cc(O)c(Cl)cc34)cc2)CC1] |
| 373 | 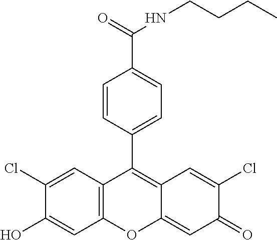 [SMILES: CCCCNC(=O)c1ccc(-c2c3cc(Cl)c(=O)cc-3oc3cc(O)c(Cl)cc23)cc1] |
| 377 | 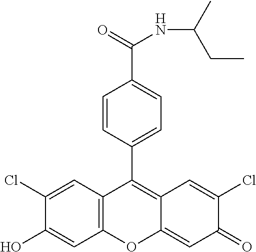 [SMILES: CCC(C)NC(=O)c1ccc(-c2c3cc(Cl)c(=O)cc-3oc3cc(O)c(Cl)cc23)cc1] |
| 382 | 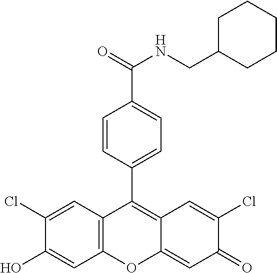 [SMILES: O=C(NCC1CCCCC1)c1ccc(-c2c3cc(Cl)c(=O)cc-3oc3cc(O)c(Cl)cc23)cc1] |
| 384 | 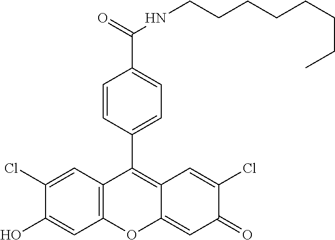 [SMILES: CCCCCCCCNC(=O)c1ccc(-c2c3cc(Cl)c(=O)cc-3oc3cc(O)c(Cl)cc23)cc1] |
| 387 | 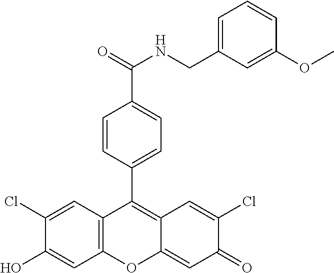 [SMILES: COc1cccc(CNC(=O)c2ccc(-c3c4cc(Cl)c(=O)cc-4oc4cc(O)c(Cl)cc34)cc2)c1] |
| 395 | 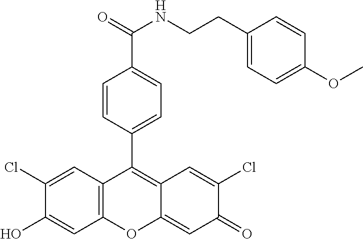 [SMILES: COc1ccc(CCNC(=O)c2ccc(-c3c4cc(Cl)c(=O)cc-4oc4cc(O)c(Cl)cc34)cc2)cc1] |
| 419 | 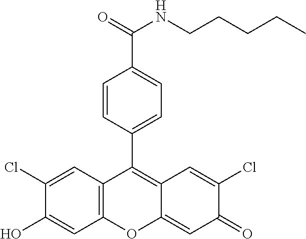 [SMILES: CCCCCNC(=O)c1ccc(-c2c3cc(Cl)c(=O)cc-3oc3cc(O)c(Cl)cc23)cc1] |
| 422 | 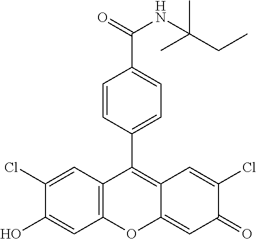 [SMILES: CCC(C)(C)NC(=O)c1ccc(-c2c3cc(Cl)c(=O)cc-3oc3cc(O)c(Cl)cc23)cc1] |
| 424 | 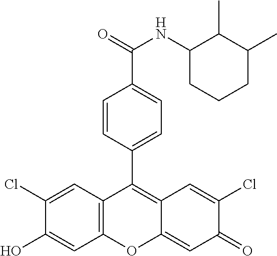 [SMILES: CC1CCCC(NC(=O)c2ccc(-c3c4cc(Cl)c(=O)cc-4oc4cc(O)c(Cl)cc34)cc2)C1C] |
| 426 | 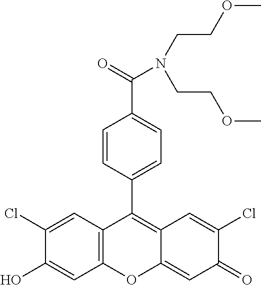 [SMILES: COCCN(CCOC)C(=O)c1ccc(-c2c3cc(Cl)c(=O)cc-3oc3cc(O)c(Cl)cc23)cc1] |
| 428 | 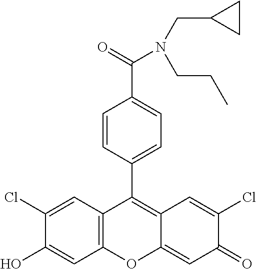 [SMILES: CCCN(CC1CC1)C(=O)c1ccc(-c2c3cc(Cl)c(=O)cc-3oc3cc(O)c(Cl)cc23)cc1] |
| 429 | 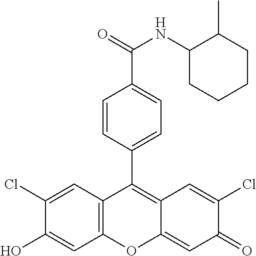 [SMILES: CC1CCCCC1NC(=O)c1ccc(-c2c3cc(Cl)c(=O)cc-3oc3cc(O)c(Cl)cc23)cc1] |
| 439 | 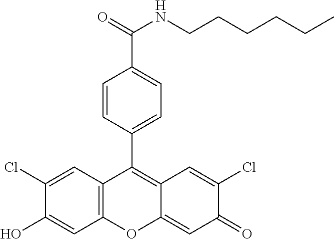 [SMILES: CCCCCCNC(=O)c1ccc(-c2c3cc(Cl)c(=O)cc-3oc3cc(O)c(Cl)cc23)cc1] |
| 441 | 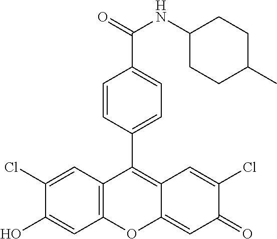 [SMILES: CC1CCC(NC(=O)c2ccc(-c3c4cc(Cl)c(=O)cc-4oc4cc(O)c(Cl)cc34)cc2)CC1] |
| 442 | 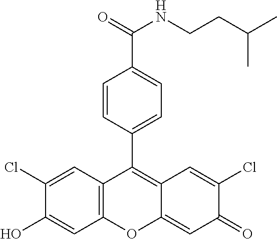 [SMILES: CC(C)CCNC(=O)c1ccc(-c2c3cc(Cl)c(=O)cc-3oc3cc(O)c(Cl)cc23)cc1] |
| 443 | 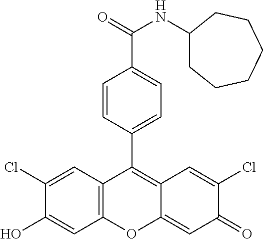 [SMILES: O=C(NC1CCCCCC1)c1ccc(-c2c3cc(Cl)c(=O)cc-3oc3cc(O)c(Cl)cc23)cc1] |
| 462 | 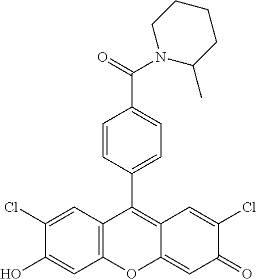 [SMILES: CC1CCCCN1C(=O)c1ccc(-c2c3cc(Cl)c(=O)cc-3oc3cc(O)c(Cl)cc23)cc1] |
| 477 | 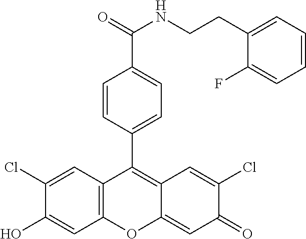 [SMILES: O=C(NCCc1ccccc1F)c1ccc(-c2c3cc(Cl)c(=O)cc-3oc3cc(O)c(Cl)cc23)cc1] |
| 478 | 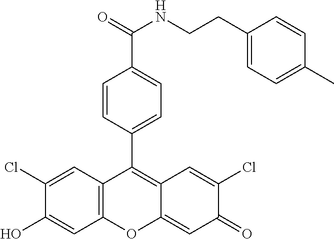 [SMILES: Cc1ccc(CCNC(=O)c2ccc(-c3c4cc(Cl)c(=O)cc-4oc4cc(O)c(Cl)cc34)cc2)cc1] |
| 480 | 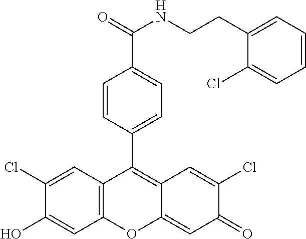 [SMILES: O=C(NCCc1ccccc1Cl)c1ccc(-c2c3cc(Cl)c(=O)cc-3oc3cc(O)c(Cl)cc23)cc1] |
| | 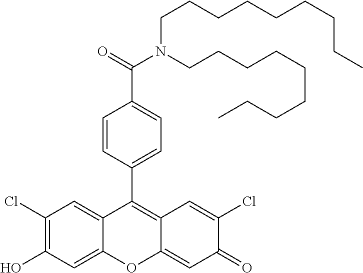 [SMILES: CCCCCCCCCN(CCCCCCCCC)C(=O)c1ccc(-c2c3cc(Cl)c(=O)cc-3oc3cc(O)c(Cl)cc23)cc1] |
| 559 | 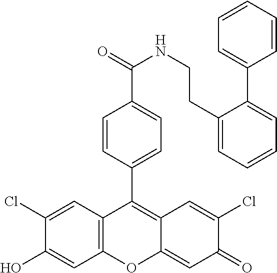 [SMILES: O=C(NCCc1ccccc1-c1ccccc1)c1ccc(-c2c3cc(Cl)c(=O)cc-3oc3cc(O)c(Cl)cc23)cc1] |
| 602 | 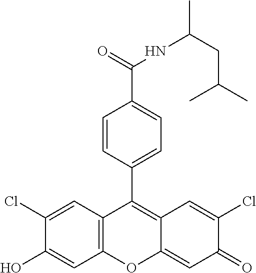 [SMILES: CC(C)CC(C)NC(=O)c1ccc(-c2c3cc(Cl)c(=O)cc-3oc3cc(O)c(Cl)cc23)cc1] |
| 657 | 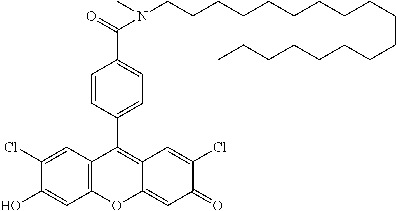 [SMILES: CCCCCCCCCCCCCCCCCCN(C)C(=O)c1ccc(-c2c3cc(Cl)c(=O)cc-3oc3cc(O)c(Cl)cc23)cc1] |

TABLE 3
Spectroscopic Properties of CF Library

| Plate | | | | Plate | | | |
|---|---|---|---|---|---|---|---|
| Code | Abs (nm) | Em (nm) | QY (Φ) | Code | Abs (nm) | Em (nm) | QY (Φ) |
| CF-A2 | 520 | 558 | 0.42 | CF-C5 | 520 | 558 | 0.63 |
| CF-A3 | 520 | 559 | 0.40 | CF-C6 | 520 | 554 | 0.58 |
| CF-A4 | 520 | 558 | 0.26 | CF-C7 | 520 | 558 | 0.42 |
| CF-A5 | 520 | 556 | 0.75 | CF-C8 | 520 | 558 | 0.68 |
| CF-A6 | 520 | 557 | 0.43 | CF-C9 | 520 | 558 | 0.50 |
| CF-A7 | 520 | 560 | 0.58 | CF-C10 | 520 | 558 | 0.54 |
| CF-A8 | 520 | 558 | 0.37 | CF-C11 | 520 | 553 | 0.82 |
| CF-A9 | 520 | 557 | 0.63 | CF-D2 | 520 | 557 | 0.61 |
| CF-A10 | 520 | 558 | 0.63 | CF-D3 | 520 | 555 | 0.47 |
| CF-A11 | 520 | 558 | 0.36 | CF-D4 | 520 | 555 | 0.69 |
| CF-B2 | 520 | 558 | 0.53 | CF-D5 | 520 | 555 | 0.66 |
| CF-B3 | 520 | 557 | 0.32 | CF-D6 | 520 | 555 | 0.32 |
| CF-B4 | 520 | 556 | 0.90 | CF-D7 | 520 | 555 | 0.54 |
| CF-B5 | 520 | 559 | 0.54 | CF-D8 | 520 | 558 | 0.47 |
| CF-B6 | 520 | 558 | 0.77 | CF-D9 | 520 | 558 | 0.25 |
| CF-B7 | 520 | 556 | 0.70 | CF-D10 | 520 | 558 | 0.59 |
| CF-B8 | 520 | 559 | 0.57 | CF-D11 | 520 | 558 | 0.44 |
| CF-B9 | 520 | 558 | 0.60 | CF-E2 | 520 | 557 | 0.63 |
| CF-B10 | 520 | 559 | 0.56 | CF-E3 | 520 | 554 | 0.66 |
| CF-B11 | 520 | 558 | 0.66 | CF-E4 | 520 | 558 | 0.47 |
| CF-C2 | 520 | 559 | 0.78 | CF-E5 | 520 | 556 | 0.58 |
| CF-C3 | 520 | 559 | 0.37 | CF-E6 | 520 | 557 | 0.29 |
| CF-C4 | 520 | 559 | 0.55 | CF-E7 | 520 | 557 | 0.46 |
| CF-F8 | 520 | 559 | 0.34 | CF-G10 | 520 | 558 | 0.23 |
| CF-F9 | 520 | 557 | 0.34 | CF-G11 | 520 | 557 | 0.35 |
| CF-F10 | 520 | 559 | 0.35 | CF-H2 | 520 | 556 | 0.65 |
| CF-F11 | 520 | 559 | 0.21 | CF-H3 | 520 | 553 | 0.82 |
| CF-G2 | 520 | 553 | 0.85 | CF-H4 | 520 | 556 | 0.63 |
| CF-G3 | 520 | 552 | 0.63 | CF-H5 | 520 | 557 | 0.67 |
| CF-G4 | 520 | 556 | 0.40 | CF-H6 | 520 | 553 | 0.65 |
| CF-G5 | 520 | 557 | 0.38 | CF-H7 | 520 | 557 | 0.59 |
| CF-G6 | 520 | 556 | 0.29 | CF-H8 | 520 | 556 | 0.51 |
| CF-G7 | 520 | 559 | 0.41 | CF-H9 | 520 | 558 | 0.30 |
| CF-G8 | 520 | 556 | 0.29 | CF-H10 | 520 | 557 | 0.38 |
| CF-G9 | 520 | 557 | 0.27 | CF-H11 | 520 | 552 | 0.32 |

Absorbance and fluorescence excitation and emission data were recorded by a Synergy 4, Biotek Inc. fluorescent plate reader in 96-well polypropylene plates (sample concentration: 100 μM in ethanol). Quantum yield (Φ) are calculated using the following equation, Φ_{CF} = Φ_{ref} (E_{CF}/E_{ref}) (η_{CF}²/η_{ref}²) (A_{ref}/A_{CF}); where Φ_{ref} is known value of reference (fluorescein), E is the integrated emission spectrum, A is the absorbance at the excitation wavelength, and η is the refractive index of the solvents used.

Synthetic Procedures

### Methyl 4-(bis(5-chloro-2,4-dihydroxyphenyl)methyl)benzoate (Compound 1)

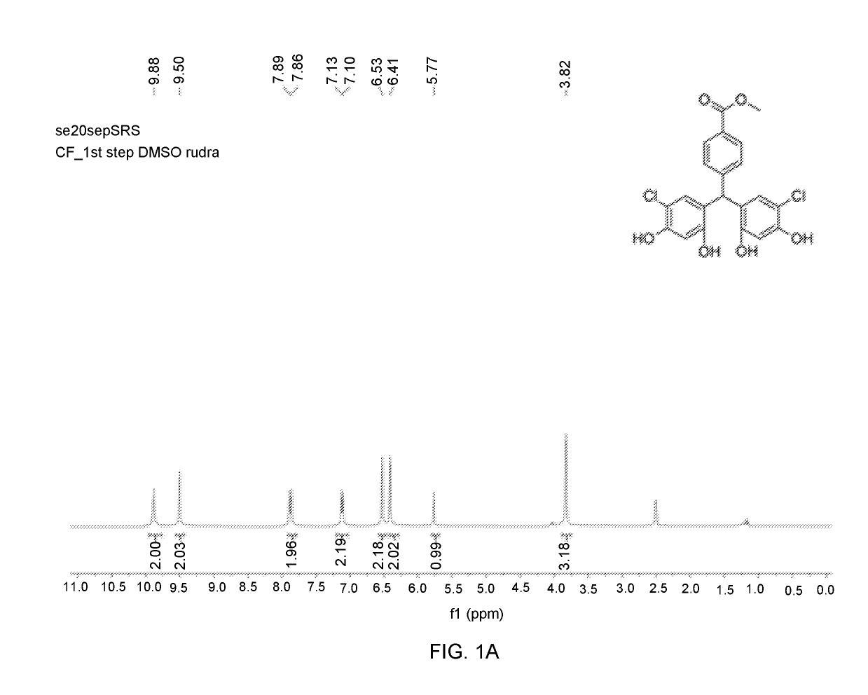

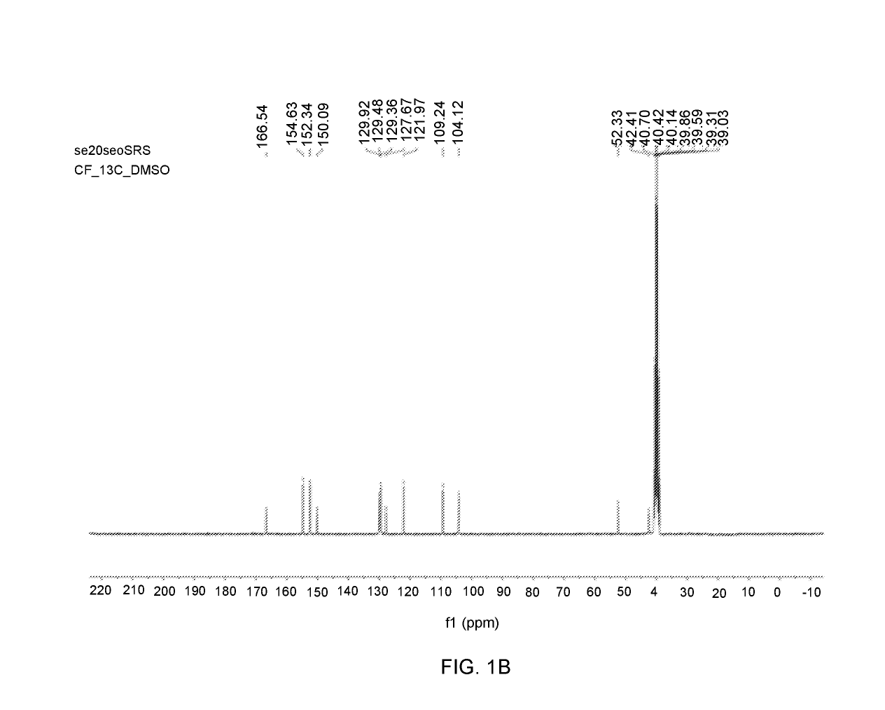

Methyl 4-formylbenzoate (0.82 g, 5 mmole) and 4-chlorobenzene-1,3-diol (1.45 g, 10 mmole) were dissolved together in DCM (20 mL). Methanesulfonic acid (2.5 mL) was added to it slowly and the reaction mixture was allowed to stir at room temperature overnight. The reaction mixture was quenched with water (20 mL) The organic layer was washed in water (10 mL) three times. Then the organic layer was dried over Na₂SO₄ and dried by rotary evaporation. Crude product was purified by silica gel column chromatography (EA:Hexane=1:4). The product was obtained as yellowish solid (2 g, 92%). ¹H NMR (300 MHz, DMSO-d₆): δ (ppm) 9.88 (s, 2H), 9.50 (s, 4H), 7.86 (d, 2H, 9 Hz), 7.13 (d, 2H, 9 Hz), 6.53 (s, 2H), 6.41 (s, 2H), 5.77 (s, 1H), 3.82 (s, 3H). ¹³C NMR (75 MHz, DMSO-d₆): δ (ppm) 166.54, 154.63, 152.34, 150.09, 129.92, 129.48, 129.36, 127.67, 121.97, 109.24, 104.12, 52.33, 42.41; EI-MS (m/z): Calc'd for C₂₁H₁₆Cl₂O₆ 434.0; found 435.0 (M+H). (FIG. 1).

### 4-(2,7-dichloro-3,6-dihydroxy-9H-xanthen-9-yl)benzoic acid (Compound 2)

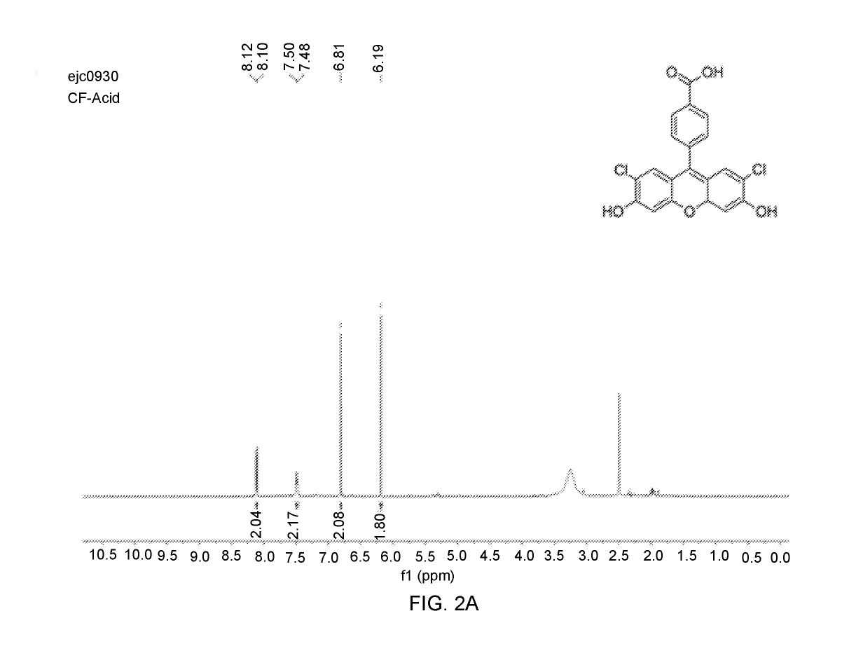

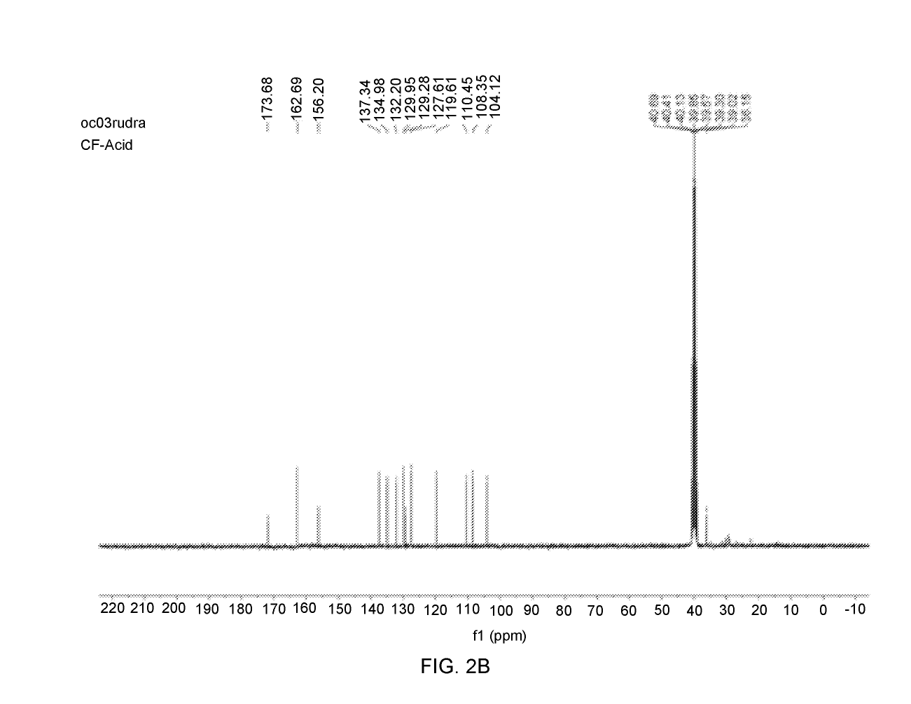

Compound 1 (22 mg, 0.05 mmol) was dissolved in toluene (5 mL). P-Toulene sulfonic acid (76 mg, 0.40 mmol) was added to it and the mixture was refluxed at 170° C. for 18 h. The reaction mixture was cooled and then quenched by saturated NaHCO₃ solution. Then the organic layer was dried over Na₂SO₄ and dried by rotary evaporation. Crude product was purified by silica gel column chromatography (MeOH:DCM=2:5). The product was obtained as reddish solid (3.2 mg, 16%). ¹H NMR (300 MHz, DMSO-d₆): δ (ppm) 8.12 (d, 2H, 8 Hz), 7.50 (d, 2H, 8 Hz), 6.81 (s, 2H), 6.19 (s, 2H). ¹³C NMR (75 MHz, DMSO-d₆): δ (ppm) 173.68, 162.69, 156.20, 137.34, 134.98, 132.20, 129.95, 129.28, 127.61, 119.61, 110.45, 108.35, 104.12, 36.15. ESI-MS (m/z): Calc'd for C₂₀H₁₂Cl₂O₅ 402.0; found 400.9 (M−H). (FIG. 2).

General Procedure for the Synthesis of CF-Library:

Compound 2 (1 eq), amine (2 eq) and HBTU (2.5 eq) dissolved in DCM/DMF (4/1). DIEA (2.5 eq) was added to the reaction mixture. The reaction mixture was stirred in rt until completion of the reaction. Product was purified by column chromatography using methanol and dichloromethane as eluent. All the library compounds were characterised by LC-MS. The spectral data of library compounds are summarized in Table 3.

Characterization of CDg13:

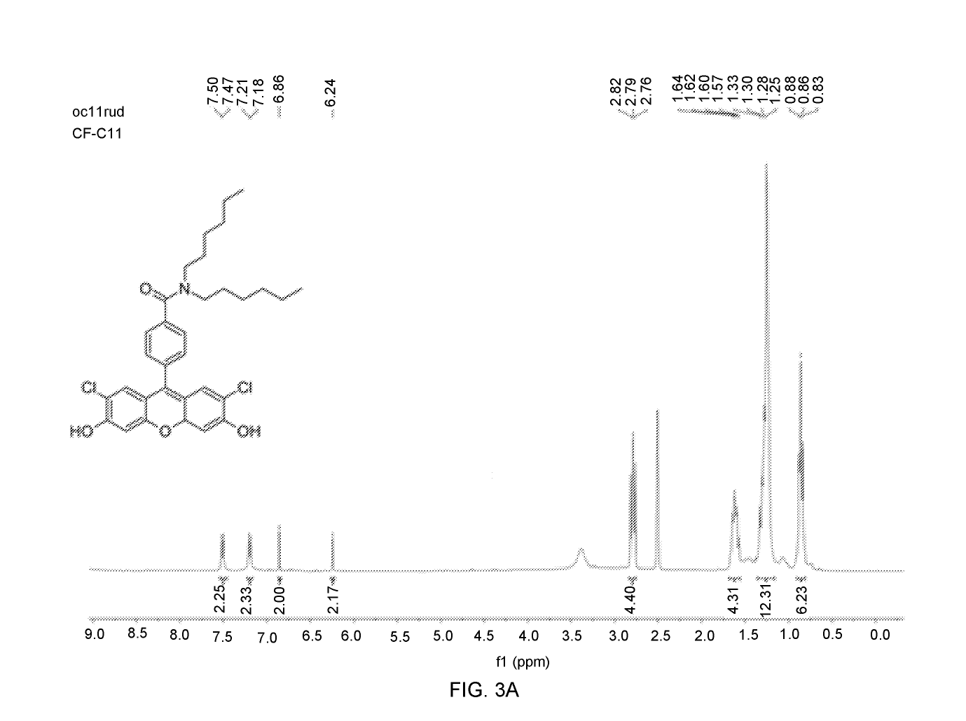

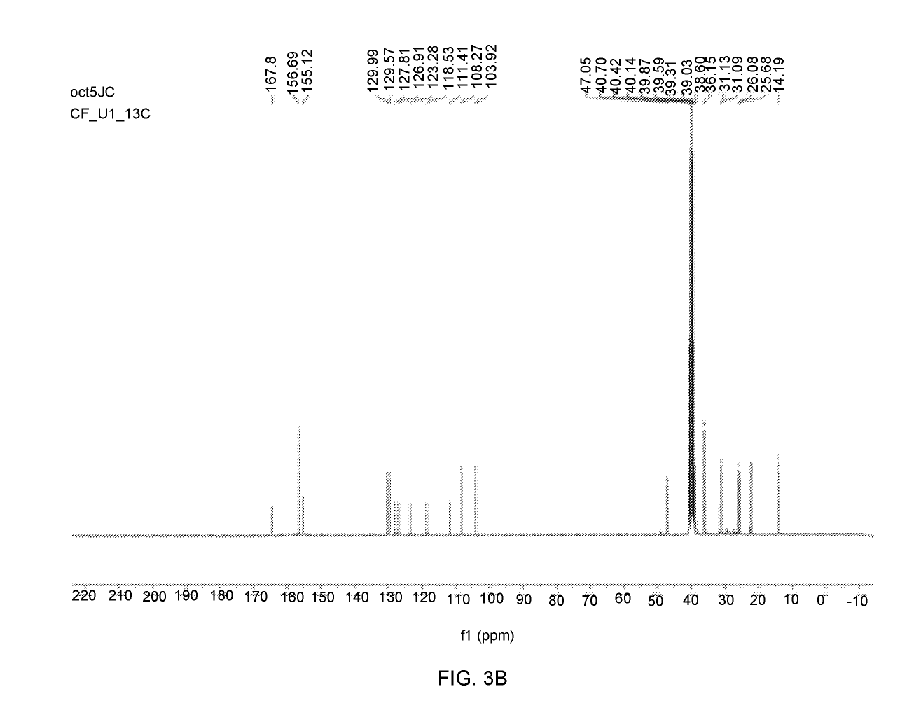

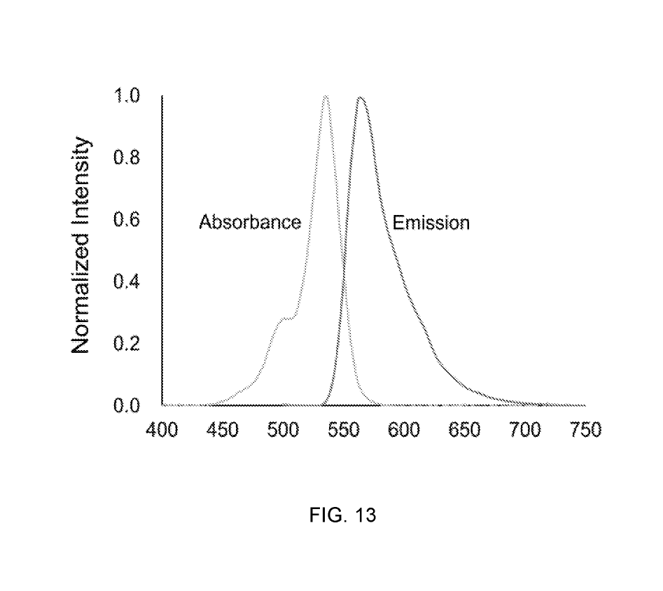

The product was obtained as red solid. ¹H and ¹³C NMR Spectra of CDg13 were as follows (FIG. 3): ¹H NMR (300 MHz, DMSO-d₆): δ (ppm) 7.50 (d, 2H, 9 Hz), 7.21 (d, 2H, 9 Hz), 6.86 (s, 2H), 6.24 (s, 2H), 2.79 (t, 4H, 9 Hz), 1.60 (m, 4H), 1.28 (m, 12H), 0.86 (t, 6H, 7.5 Hz). ¹³C NMR (75 MHz, DMSO-d₆): δ (ppm) 167.18, 156.69, 155.12, 129.99, 129.57, 127.81, 126.91, 123.28, 118.53, 111.41, 108.27, 103.93, 47.05, 36.15, 31.13, 26.08, 25.68, 14.19. HRMS: m/z calc'd for C₃₂H₃₅Cl₂NO₄ (M+H)+568.2016; found 568.2023. λ_{abx}/λₑₘ=520/553 nm and quantum yield=0.82, extinction co-efficient of CDg13=40933.2 M⁻¹ cm⁻¹ measured in ethanol. Normalized absorption and emission spectra of CDg13 can be seen in FIG. 13.

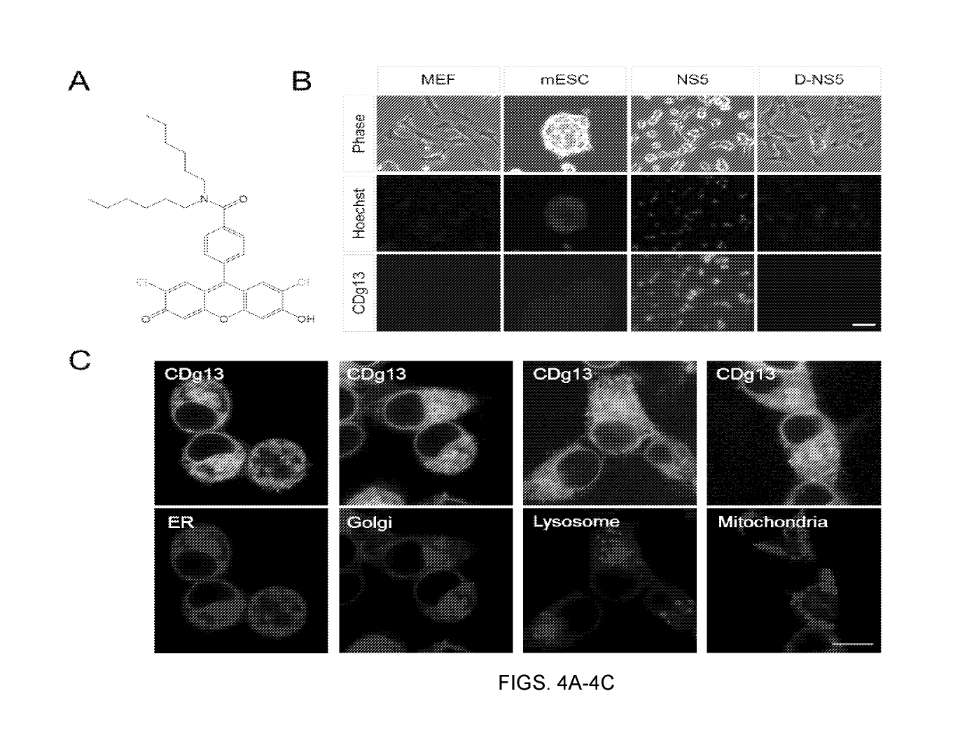

The screening platform was composed of mouse embryonic fibroblast (MEF), mouse embryonic stem cells (mESC), NS5 and differentiated NS5 (D-NS5) (FIG. 4). A derivate from the CF library was selected having eleven saturated carbon chain (CF-C11), named as compound of designation green 13 (CDg13, λ_{abs}/λₑₘ=520/553 nm; quantum yield=0.82) (FIG. 4A, Table 3). CDg13 preferentially stains undifferentiated NS-5 cells, a NSPC line, but not other cell types (FIG. 4B). To characterize the subcellular target of the CDg13 probe, the image of CDg13 using confocal microscopy was analyzed. The CDg13 stained organelle was clearly merged with the staining of a molecular probe that targeted endoplasmic reticulum (ER), but not with Golgi, lysosome, and mitochondria targeted probes (FIG. 4C). The value of co-localization with ER was appeared as 0.97 on both of Pearson's Collection and Mander's overlap, whereas other organelle markers were below 0.83.

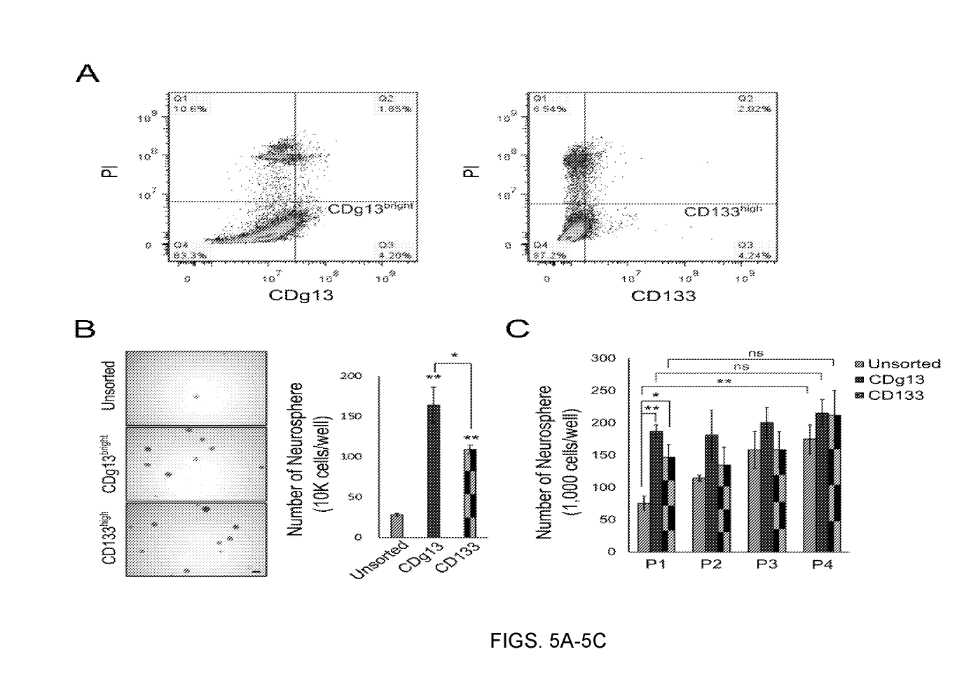

Next, whether the small chemical probe, CDg13, can be applied to isolate RG from E14.5 mouse forebrains was analyzed. RG are NSPCs of embryonic brain, and they form neurospheres under in vitro condition with bFGF and EGF. A population of cells from E14.5 mouse embryonic brain was isolated using FACS with staining of CDg13 or CD133/Prominin antibody—the most well-known surface marker for neural stem cell isolation (FIG. 5A). Cells with bright CDg13 fluorescence (CDg13^{bright}) formed enhanced number of neurospheres compared to that from unsorted brain cells (5.9-fold) and to that from CD133 positive cells (CD133^{high}) (1.5-fold) (FIG. 5B). Although the enhanced number of neurospheres were observed, there was a possibility that the enrichment of neurosphere forming cells might be derived by intermediate progenitor cells (IPCs) since they also can generate primary neurosphere with their limited proliferation capacity. To test NSPC property, neurospheres with same number of cells (1,000 cells/well) were passaged to analyze their self-renewal potential until passage 4. The cells derived from CDg13 or CD133 sorted neurospheres formed maximum number of daughter neurospheres from passage 1 to 4 with similar levels, indicating that they sustained their self-renewal capacity (FIG. 5C). Whereas, the neurosphere forming capacity of cells from unsorted neurospheres was gradually increased by passage number, and reached to the levels of CDg13 and CD133 sorted neurospheres at around 3-4 passage because of elimination of IPCs (FIG. 5C). These data implicated that CDg13 isolates self-renewable NSPCs directly from mouse embryonic brain as much as CD133 immunostaining.

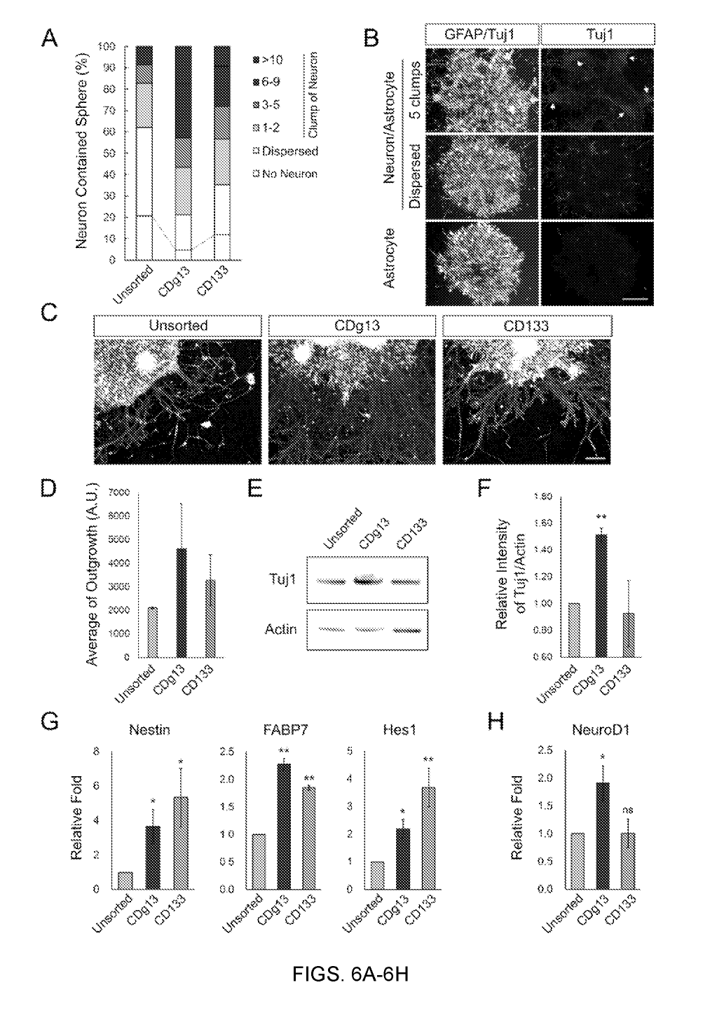

NSPCs have potential to differentiate into neuron and glia. To analyze the differentiation potential of CDg13^{bright} cells, primary neurospheres were randomly differentiated using serum-containing media on poly-D-lysine coated culture vessels. The differentiated cells were immunostained with Tuj1 and GFAP, markers of neuron and astrocyte, respectively (FIG. 6). Interestingly, the neurospheres formed from CDg13^{bright} cells were highly differentiated to neuron compared to the neurospheres from unsorted and CD133^{high} cells, 95.3% (CDg13) versus 79.3% (Unsorted) and 88.0% (CD133) in the whole spheres (FIG. 6A). To further clarify their neurogenic potential, the number of neuron clumps was counted, often observed in multi-potent neurospheres (FIG. 6B). A neuron clump was defined if there were more than 10 of neuronal cell bodies aggregated to each other (FIG. 6B). The multi-potent neurospheres originated from CDg13^{bright} cells contained more neuron clumps than that from other groups, suggesting that more neurons were differentiated in the neurospheres derived from CDg13^{bright} cells (FIGS. 6A, 6B). However, average neurite outgrowth of neurons from CDg13^{bright} neurospheres was more, but not as significant developed as that from unsorted and CD133^{high} cells-sorted neurospheres, indicating that neurons from all groups were developed similar levels (FIGS. 6C, 6D). The quantification of total Tuj1 levels from differentiated neurospheres supported that the more neurons were differentiated from neurospheres derived from CDg13^{bright} cells (FIGS. 6E, 6F).

To further evaluate the characteristic of CDg13^{bright} NSPCs, gene expression of NSPC markers was analyzed. Three markers for NSPCs, Nestin, FABP-7/BLBP, and Hest, were significantly enhanced in the enriched NSPCs using CDg13 probe and CD133 antibody (FIG. 6G). It supported the fact that the two groups have NSPC property. Interestingly, NeuroD1, neurogenic IPC marker gene, was significantly increased only in CDg13^{bright} cell population but not in the CD133^{high} cell population (FIG. 6H). This information suggested that CDg13^{bright} NSPCs has unique properties with different gene expression of NeuroD1 compared to the CD133^{high} NSPCs.

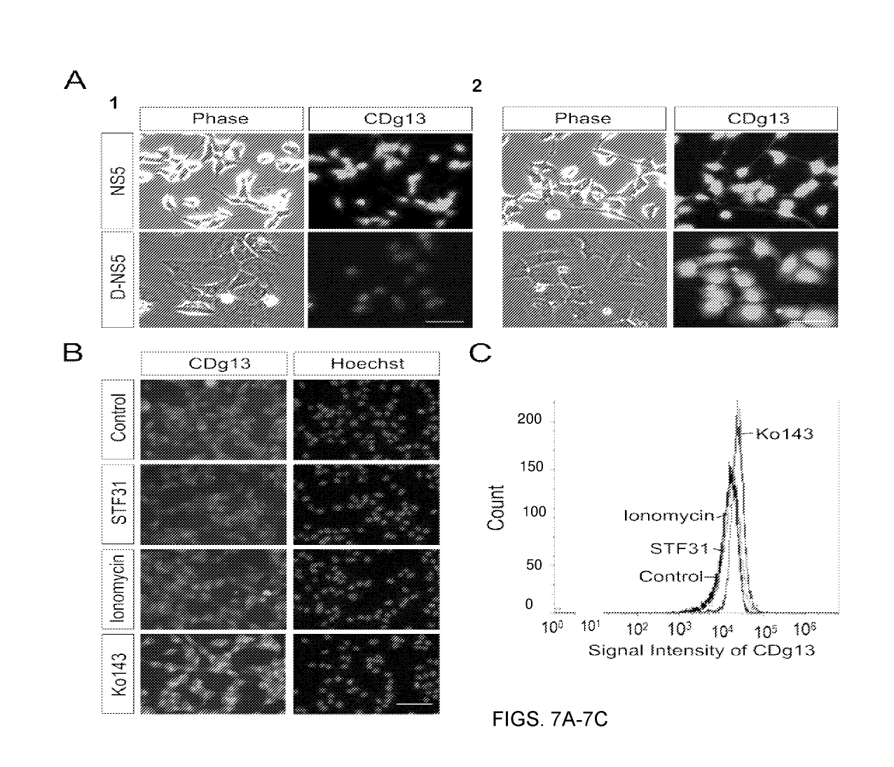

To examine the mechanism of CDg13 staining, several approaches were performed. Since CDg13 non-specifically stained dead cells either in NS-5 and differentiated NS-5 (FIG. 7A), it was hypothesized that most live cells block to enter the compound into cells or actively secrete the compound to extracellular space. First, neither inhibition of the NSPC specific channel, glucose transporter 1 (Glut1) (STF31), nor disruption of membrane potential by using calcium ionophore (ionomycin) reduced CDg13 staining in NS-5 cells, indicating that entrance of the compound is not mediated by inward channel or their membrane property (FIGS. 7B and 7C). Interestingly, however, blocking secretion mechanism through treatment of a specific Abcg2 inhibitor, Ko143, stained CDg13 more strongly than normal NS-5 cells (FIGS. 7B and 7C).

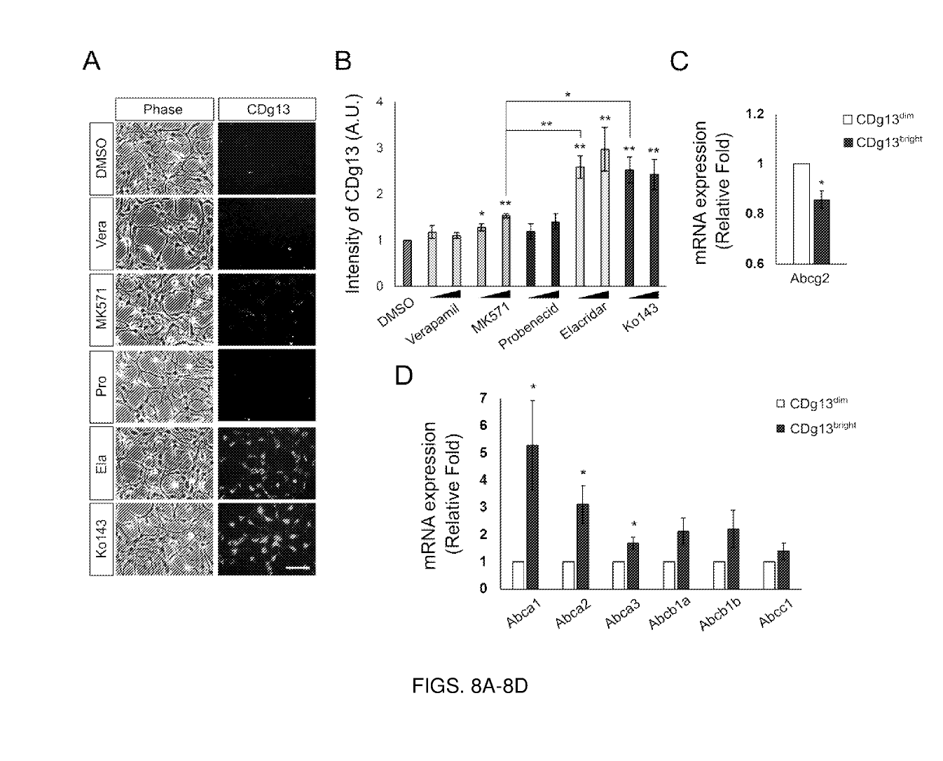

Various inhibitors of ABC transporters were tested to analyze the specificity of CDg13 staining as several ABC transporters mediate stem cells or cancer cells capacity. Verapamil, MK571, probenecid, elacridar and Ko143 were used to block Abcb1, Abcc1-4, Abcb1/Abcg2 and Abcg2, respectively on differentiated NS5 cells. As a result, verapamil and probenecid had no effect of probe staining. Elacridar and Ko143 significantly increased the staining of CDg13 around 2.5 fold than DMSO control (FIGS. 8A-B). Although MK571 also affected CDg13 staining, the intensity of staining was much lower (˜1.5 fold) and had significance with the value of elacridar and Ko143 (FIGS. 8A-B). The involvement of Abcg2 on the staining of CDg13 was also supported by gene expression analysis with FACS sorted cells using CDg13 from cultured neurospheres. By comparing of ABC transporters mRNA between the bright (CDg13^{bright}) and dim (CDg13^{dim}) CDg13 contained cells, only Abcg2 expression was significantly decreased in CDg13^{bright} cells was observed (FIG. 8C). All other ABC transporters that were tested, Abca1, Abca2, Abca3, Abcb1a, Abcb1b and Abcc1, were similarly or even highly expressed in CDg13^{bright} than CDg13^{dim} cells (FIG. 8D).

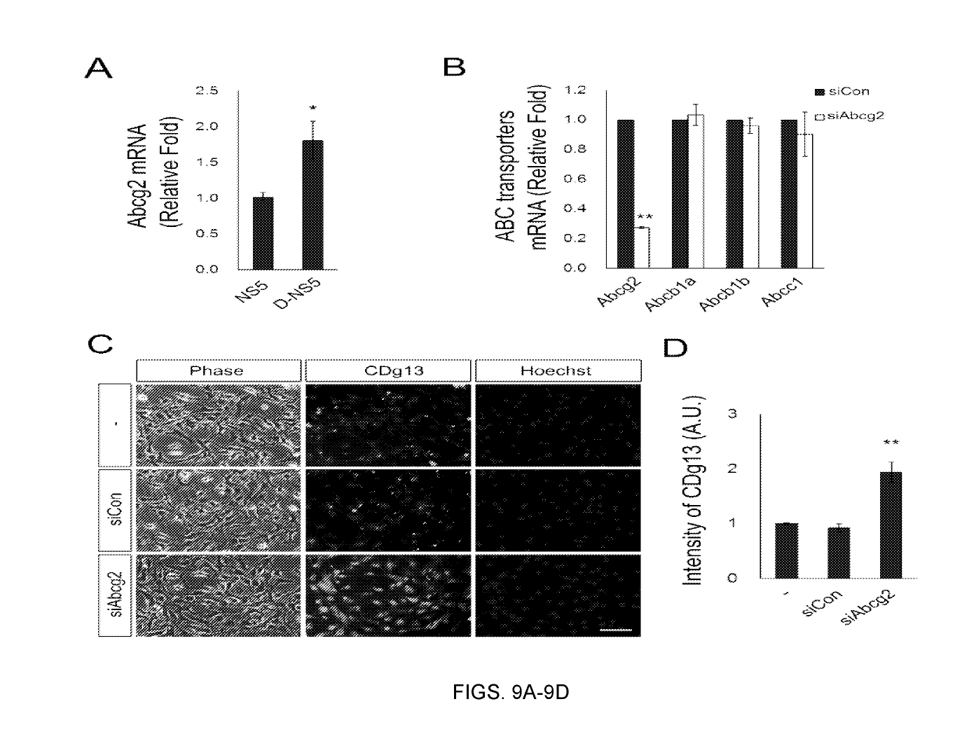

Hence, the expression of Abcg2 was also increased in astrocytes (D-NS5) compared to un-differentiated NSPC (NS5), supporting the low expression of Abcg2 in NSPCs (FIG. 9A). It was further confirmed that the involvement of Abcg2 transporter for CDg13 staining by knockdown of Abcg2 using siRNA on D-NS5. After Abcg2 siRNA transfection to D-NS5 cells, Abcg2 was specifically suppressed around 23% of control levels without any influence on the other two major ABC transporters, Abcb1 and Abcc1 (FIG. 9B). In this condition, Abcg2 knockdown cells were strongly stained by CDg13 (FIG. 9C). Quantification analysis showed CDg13 staining was increased around 2-fold by the Abcg2 knockdown than the other controls (FIG. 9D).

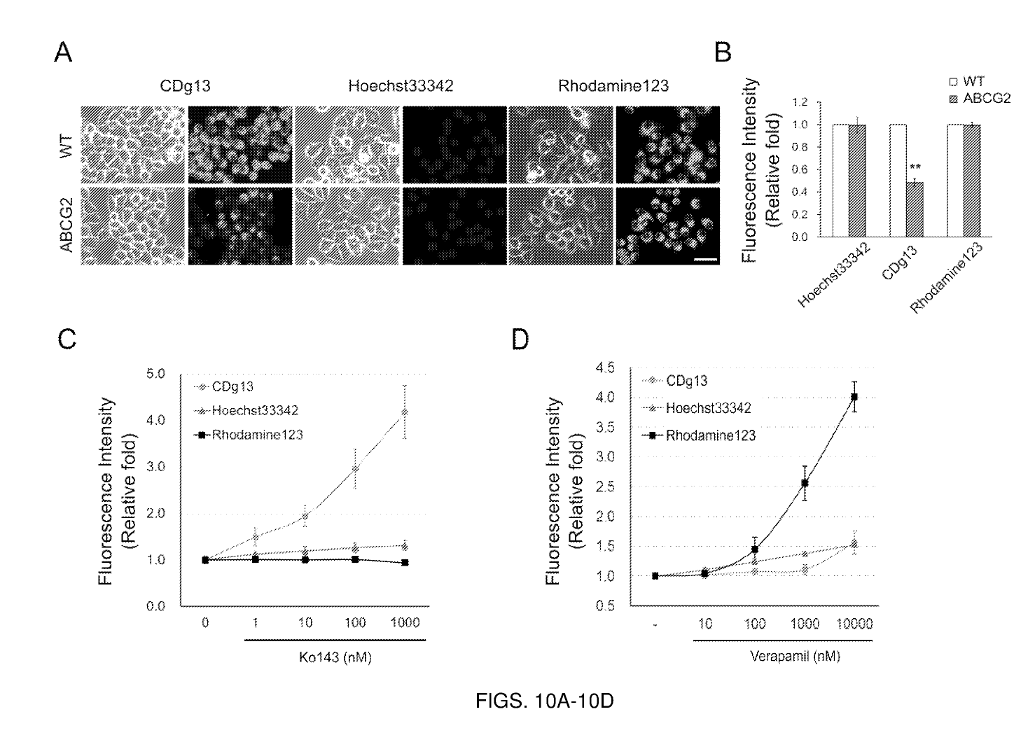

Whether CDg13 is also a substrate for human ABCG2 was tested by using ABCG2 overexpressed KB3-1 cell line (ABCG2/KB3-1). ABCG2/KB3-1 cells were poorly stained to CDg13 as compared to wild-type KB3-1 (FIGS. 10A-B). However, neither staining with Hoechst 33342 nor Rhodamine123, tracers for ABCB1 (also known as P-glycoprotein) & ABCG2 and ABCB1 respectively, was affected by overexpression of ABCG2 (FIGS. 10A-B). The uptake of CDg13 through ABCG2 was further confirmed by inhibition of ABCG2 activity using Ko143 in ABCG2/KB3-1. The gradual increase of CDg13 signal was observed in the range of 10 to 1,000 nM of Ko143 (FIG. 10C). Although this phenomenon was also observed in Hoechst 33342, the fold change in fluorescence intensity was largely elevated in CDg13 (up to 4.2-fold) as compared to Hoechst 33342 (up to 1.3-fold) (FIG. 10C). As expected, fluorescence signal of Rhodamine123 did not increase (FIG. 10C). The effect of ABCB1 inhibition was also examined by using HCT-15, which was reported as the cell line retaining the highest level of human ABCB1 among NCI-60 cell lines⁵. The treatment of 10 to 10,000 nM verapamil, an ABCB1 inhibitor, enhanced staining of Rhodamine123 (up to 4.0 fold) and Hoechst 33342 (up to 1.5 fold) (FIG. 10D). However, no changes were observed in CDg13, unless treated with the highest amount of verapamil (1.5 fold induction in 10 μM of verapamil) (FIG. 10D). These suggest that CDg13 has higher sensitivity and selectivity to human ABCG2 as compared to the well-known fluorescent probe, Hoechst 33342.

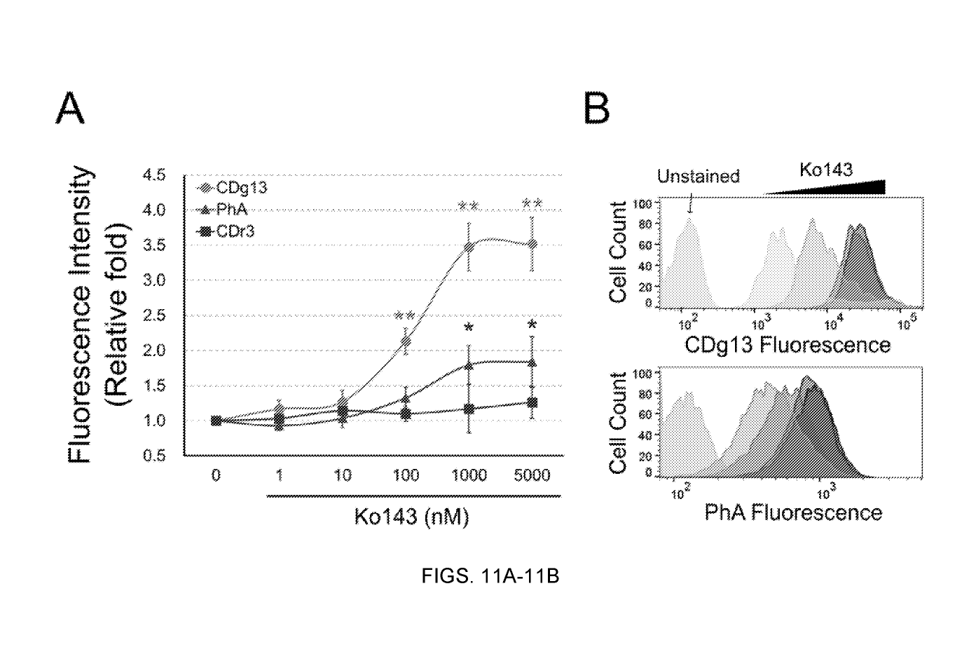

Currently, the chlorophyll catabolite, pheophorbide A (PhA) is the only ABCG2 specific fluorescent substrate⁶. When the response of the CDg13, CDr3 and PhA was compared to ABCG2, CDg13 showed higher sensitivity and produced more consistent data than PhA (FIG. 11). Moreover, the detection of the fluorescence of CDg13 is easily accessible because of the use of standard fluorescein filter as compared to the specific spectrum of fluorescence for PhA (λex/λem=635/561 or 488/670 nm)⁶.

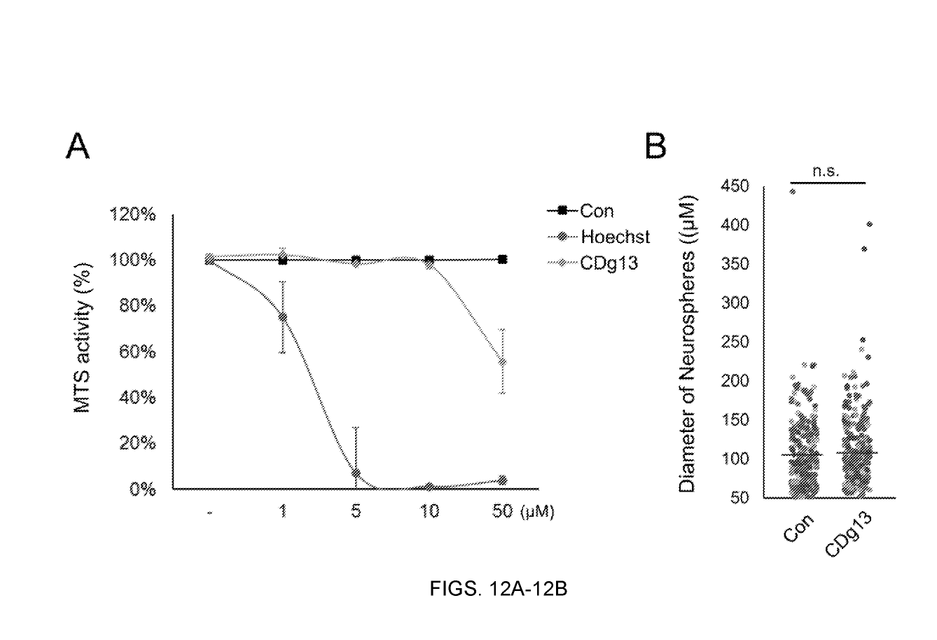

The toxicity of CDg13 to both human and mouse cells through MTS assay was examined next. No toxicity was observed on human KB3-1 cells between 1 to 10 μM of CDg13 during 48 h. Cells started dying at 50 μM of CDg13, 50 times more concentrated than our working concentration (FIG. 12A). However, treatment with 1 μM of Hoechst 33342 resulted in a death rate of 25% and further increase to 10 μM resulted in 100% cell death. This means that CDg13 has very low toxicity to cells under its working concentration. The effect of CDg13 on the proliferation of mouse NSPCs was also tested. Co-incubation of mouse NSPCs with 1 μM of CDg13 for 7 days showed no significant difference in neurosphere size, 106 versus 109 μm on average for control and CDg13-containing neurospheres at third passage. This suggests that CDg13 does not have any effect on the proliferation of NSPCs even in long-term culture condition (FIG. 12B). It was concluded that the ABCG2-specific fluorescent substrate, CDg13, selectively stains a population of NSPCs having lower levels of Abcg2, and the NSPCs have higher capacity to form neurons.

Reagents:

All the chemicals and solvents were purchased from Sigma Aldrich, Alfa Aesar, Fluka, MERCK, Tocris or Acros, and used without further purification. Normal phase purifications were carried out using Merck Silica Gel 60 (particle size: 0.040-0.063 mm, 230-400 mesh). Analytical characterization was performed on a HPLC-MS (Agilent-1200 series) with a DAD detector and a single quadrupole mass spectrometer (6130 series) with an ESI probe. ¹H-NMR and ¹³C-NMR spectra were recorded on Bruker Avance 300 MHz NMR spectrometers, and chemical shifts are expressed in parts per million (ppm) and coupling constants are reported as a J value in Hertz (Hz). High resolution mass spectrometry (HRMS) data was recorded on a Micro mass VG 7035 (Mass Spectrometry Laboratory at National University of Singapore (NUS)). Spectroscopic and quantum yield data were measured on spectroscopic measurements, performed on a fluorometer and UV/VIS instrument, Synergy 4 of Bioteck Company. The slit width was 1 nm for both excitation and emission, and the data analysis was performed using GraphPrism 5.0.

Cell Culture:

Mouse embryonic stem cells (mESCs) were cultured on gelatin-coated culture plate with high-glucose DMEM supplemented with 20% ES FBS (v/v), 2 mM L-glutamine, 100 U/ml penicillin, 100 μg/ml streptomycin, 0.1 mM non-essential amino acids, 0.1% β-mecaptoethanol (v/v) and 100 U/mL leukemia inhibitory factor (Chemicon). Mouse embryonic fibroblast (MEF) were obtained from E14.5 mouse embryo removed brain and liver. The embryo were chopped into small pieces with scissors, and digested with trypsin/EDTA and DNase I (0.1 mg/ml, Roche diagnostic). The cells were plated in high-glucose DMEM supplemented with 10% FBS (v/v), 100 U/ml penicillin, 100 μg/ml streptomycin and 2 mM L-glutamine overnight. The attached MEF were passaged, and used within passage 4. NS5 cell was cultured in Euromed-N medium (Euroclone) supplemented with modified N2 supplements [apo-transferin (100 μg/ml, Sigma), sodium selenite (5.2 ng/ml, Sigma), progesterone (19.8 ng/ml, Sigma), putrescine (16 μg/ml, Sigma), insulin (25 μg/ml, Sigma), BSA (50.25 μg/ml)], 10 ng/ml bFGF, 10 ng/ml EGF, 100 U/ml penicillin, 100 μg/ml streptomycin and 2 mM L-glutamine. Differentiation of NS5 cells into astrocytes were achieved by incubating the cells with 5% FBS-containing Euromed-N medium more than 3 days. Inhibitors of ABC transporters, verapamil, MK571, probenecid, elacridar and Ko143, were from Tocris. All the cell culture components were from Invitrogen unless otherwise indicated.

High Throughput Screening Using DOFLA:

Screening of fluorescent probes of DOFL was conducted using high-throughput imaging analysis as previously described⁷.

Probes Staining:

Hoechst 33342 (2 μM) and CDg13 (1 μM) were added to the cell culture medium. After incubation for 1 hour, culture media were changed with BSA-free medium to maintain CDg13 staining. The organelle specific chemical probes, ER-Tracker™ Red, BODIPY® TR Ceramide, LysoTracker® Red DND-99 and MitoTracker® Deep Red FM, were used to stain endoplasmic reticulum, Golgi apparatus, lysosome and mitochondria, respectively according to manufacturer's instructions (Molecular Probe). The subcellular staining was observed under confocal microscope and their co-localizations were analyzed by Pearson's Collection and Mander's overlap using NIS-Elements software of Eclipse Ti microscope (Nikon).

Primary Neurosphere Culture and Differentiation:

All animal experiments were approved by the Biomedical Research Council Singapore, Institutional Animal Care and Use Committee (IACUC). E14.5 embryos were obtained from C57BL/6 pregnant mice. Cerebral cortices were removed and triturated into single-cell suspension by digestion of dissected tissues with StemPro® Accutase® (Invitrogen) and filtered through 40 μm nylon mesh. Dissociated cells were seeded at a density of 1×10³ cells/cm² in neurosphere growth medium [DMEM/F12 supplemented with 2% B27 (without vitamin A), bFGF (10 ng/ml), EGF (20 ng/ml), 1× anti-anti]. All the cell culture components were from Invitrogen. Passaging of neurosphere was conducted through single cell dissociation of neurospheres as described above. Single cells were then incubated with neurosphere growth medium at 37° C., 5% CO₂. Passaging was performed every 7 days after culture. For differentiation, poly-D-lysine (Sigma) coated culture surface were used to attach neurosphere. Differentiation was induced for 6 days using the medium containing DMEM/F12 supplemented with 5% FBS, 1×B27 and 1× anti-anti.

Confocal Microscopy:

NS-5 cells stained with CDg13, Hoechst33342 and/or organelle markers were observed using A1R+si confocal microscope (Nikon) within 1 hour after staining. The live NS-5 cells were loaded into a pre-heated plate with supplemented 5% CO₂. Fast scanning less than 250 ms with 4 times scan were used to prevent phototoxicity onto the cells.

Measurement of Neurosphere Number:

For the counting of the number of neurosphere, we selected 6 days-cultured neurospheres having larger than 50 μm of diameter. The whole neurospheres in a well were counted to reduce random counting error using EVOS microscope (Advanced Microscopy Group).

Flow Cytometry:

Flow cytometry was performed using Attune Cytometer (Invitrogen). Hoechest33342 (2 μM) and CDg13 (1 μM) are incubated with culture media for 1 hours and detached as single cells. The collected cells were suspended in BSA- and FBS-free DMEM to prevent loss of CDg13 signal. The average fluorescence intensity of total cells in each experimental group was analyzed by Attune cytometer software for quantification study.

Isolation of CDg13^{bright} Neural Stem Cells:

E14.5 embryos were obtained from C57BL/6 pregnant mice. Cerebral cortices were removed and triturated into single-cell suspension by digestion of dissected tissues with StemPro® Accutase® (Invitrogen) and filtered through 40 μm nylon mesh. The brain cells were collected by centrifugation with 400×g for 3 min and resuspended in neurosphere growth medium [DMEM/F12 supplemented with 2% B27 (without vitamin A), bFGF (10 ng/ml), EGF (20 ng/ml), 1× anti-anti]. The cells were stained for 1 hour with 1 μM of CDg13 in neurosphere growth medium. After collecting cells by centrifuge as described above, the cells were resuspended to BSA-free DMEM (phenol red free) and added propidium iodide (PI) at a concentration of 1 μg/ml to distinguish dead cells. FACS sorting was performed using the MoFlo XDP cell sorter (Beckman Coulter). Cells were sorted by pre-gating with FSC/SSC properties to exclude small debris having FSC^{low}/SSC^{low}. To isolate CDg13 stained cell population, we used a 488 nm laser excitation and a 529/28 BP filter to collect emitted light. Dead cells stained by PI were detected with a 488 nm excitation and a 620/29 BP emission. We collected the cells having 10% highest CDg13 signal (CDg13^{bright}) and lower level of PI (PI^{dim}) population as illustrated in FIG. 5A. CD133/Prominin-1 immunostaining were performed separately by incubating brain cells with CD133 antibody (Biolegend, 1:50) for 1 hour, followed by secondary antibody conjugated to Alexa Fluor 488 (Invitrogen) for 30 min. The same procedure were performed to isolate CD133-positive cells after the staining procedure of CDg13. 20,000 cells of each group were collected into a tube filled with neurosphere growth medium. The cells were distributed to 6-well plate as duplicates, and cultured to form neurosphere at 37° C. in 5% CO₂.

Immunofluorescence Staining and Analysis:

More than a hundred of differentiated neurospheres were fixed in paraformaldehyde (4%, w/v) for 15 min, permeabilized in Triton X-100 (0.1% v/v), and blocked with BSA (3% w/v) for 1 hour. Neurospheres were incubated with antibodies to Tuj1/βIII-tubulin (1:500; Sigma, T5076) and GFAP (1:1,000; DAKO, Z0334) overnight at 4° C. Alexa 488-conjugated anti-mouse IgG and Cy5-conjugated anti-rabbit IgG (Invitrogen) were used to detect Tuj1 and GFAP, respectively. Nuclei were stained using Hoeschst33342 (1 μM) for 15 mins. Fluorescent images were obtained using Axio Observer microscope (Carl Zeiss). The existence of clear Tuj1 positive cells inside a differentiated neurosphere were counted as neuron-contained neurospheres. Neuronal clumps were counted if more than 10 of nuclei of neuronal cells are packaged each other. Neurite outgrowth of Tuj1 positive cells were measured using neurite outgrowth module parameter of MetaXpress (Molecular Probe) with maximum width of 1 μm. The phases with high neurite outgrowth were selected and counted at least 300 cells.

Quantitative Realtime-PCR (qRT-PCR):

RNA was extracted from 100,000-200,000 cells of live CDg13 positive or negative population using RNeasy purification kit (Qiagen). cDNA were synthesized with 100-400 ng of total RNA using Oligo dT and Superscipt III reverse transcriptase (Invitrogen). qRT-PCR were conducted with SYBR Master Mix reagents (Applied Biosystems). The expression of genes was normalized to β-actin gene expression. The information of primer sequences are as follows.

| | | | SEQ | | | |
|---|---|---|---|---|---|---|
| Gene | | | ID | | | |
| name | Dir. | Sequence | NO | Target | Gene ID | Size |
| Nestin | F | TGCTAGCCCTGCCTGTCTAC | 1 | 5975 | NM_016701.3 | 73 |
| | R | CATCATTGCTGCTCCTCTGGG | 2 | 6047 | | |
| Hest | F | ACACCGGACAAACCAAAGAC | 3 | 304 | NM_008235.2 | 147 |
| | R | ATGCCGGGAGCTATCTTTCT | 4 | 450 | | |
| Fabp7 | F | GCTTTCTGCGCAACCTGGAA | 5 | 96 | NM_021272.3 | 87 |
| | R | TTGCCTAGTGGCAAAGCCCA | 6 | 182 | | |
| NeuroD1 | F | AGCGAGTCATGAGTGCCCAG | 7 | 100 | NM_010894.2 | 86 |
| | R | GCACAGTGGATTCGTTTCCCG | 8 | 185 | | |
| Abca1 | F | TACAGTGGCGGCAACAAACG | 9 | 6453 | NM_013454.3 | 106 |
| | R | GGGCTTTAGGGTCCATGCCT | 10 | 6558 | | |
| Abca2 | F | GTCTCGGAAGATTGGCCGGA | 11 | 6276 | NM_007379.2 | 82 |
| | R | ACCAAGGAGCCCAAAGCACT | 12 | 6357 | | |
| Abca3 | F | TGCTGCCCACTACTGCAAGA | 13 | 4324 | NM_001039581.2 | 105 |
| | R | CCTGAGGCAGCCATGGAAGT | 14 | 4428 | | |
| Abcb1a | F | GGAGGCCAACATCCACCAGT | 15 | 3575 | NM_011076.2 | 136 |
| | R | GTGAGGCTGTCTGACGAGGG | 16 | 3710 | | |
| Abcb1b | F | TGGCAAAGCCGGAGAGATCC | 17 | 2474 | NM_011075.2 | 115 |
| | R | GGTCAGTGAGCCAGTGCTGT | 18 | 2588 | | |
| Abcc1 | F | CCCACCCTTGGGTCTGGTTT | 19 | 3386 | NM_008576.3 | 77 |
| | R | ACTCCAGGCGCTTCAGTTGT | 20 | 3462 | | |
| Abcg2 | F | TCACCTTACTGGCTTCCGGG | 21 | 1220 | NM_011920.3 | 107 |
| | R | CGCAGGGTTGTTGTAGGGCT | 22 | 1326 | | |
| Actin, | F | ACCAACTGGGACGACATGGAGAAG | 23 | 308 | NM_007393.3 | 214 |
| beta | R | TACGACCAGAGGCATACAGGGACA | 24 | 521 | | |

Western Blot Analysis:

Differentiated neurospheres were washed with PBS and lysed in CellLytic™ M Cell Lysis Reagent (Sigma) containing Pierce™ Protease and Phosphatase Inhibitor tablet (Thermo Scientific). Total proteins (20-30 μg) were separated by SDS-PAGE, and transferred to Immobilon®-FL PVDF membranes (Millipore). Membranes were incubated with Tuj1 (1:5,000) or β-actin (1:5,000; Santa Cruz, sc-47778), followed by incubation with Alexa 647-conjugated secondary antibodies (1:10,000). Protein bands were visualized using Typhoon 9400 Imager (GE Healthcare) and quantified with ImageQuant TL (GE Healthcare).

siRNA Transfection:

siRNAs targeted to mouse ABCG2 gene and non-targeted control (Santa Cruz) were transiently introduced to 2 days differentiated NS-5 cells by using RNAiMAX (Invitrogen). We used 20 nM of siRNA and 3 μl of RNAiMAX for transfecting one well of 12-well plate (70-80% confluence with cells). Transfection efficiency in the condition was more than 90% as measured by fluorescence non-targeted siRNA. The levels of RNA and their analysis were performed after 3 days of transfection.

### Definitions

All definitions of substituents set forth below are further applicable to the use of the term in conjunction with another substituent.

The term “alkyl,” as used herein, refers to both a saturated aliphatic branched or straight-chain monovalent hydrocarbon radical having the specified number of carbon atoms. For instance, “(C1-C6) alkyl” means a radical having from 1-6 carbon atoms in a linear or branched arrangement. Examples of “(C1-C6) alkyl” include, for example, n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl. Alkyl can be optionally substituted with halogen, —OH, oxo, (C1-C6)alkyl, (C1-C6)alkoxy, (C1-C6) alkoxy(C1-C4)alkyl, aryl, heteroaryl, heterocyclyl, cycloalkyl, carbocyclyl, nitro, cyano, amino, acylamino, or carbamyl, —C(O)O(C1-C10)alkyl, or —C(O)(C1-C10)alkyl.

The term “cycloalkyl,” as used herein, refers to saturated aliphatic cyclic hydrocarbon ring. Thus, “(C3-C8) cycloalkyl”, for example, means (3-8 membered) saturated aliphatic cyclic hydrocarbon ring. (C3-C8) cycloalkyl includes, but is not limited to, cyclopropyl, cyclobutyl, cyclopentyl, cyclohexyl, cycloheptyl, or cyclooctyl. Cycloalkyl can be optionally substituted in the same manner as alkyl, described above.

The term “amino,” as used herein, refers to a primary (—NH2), secondary (—NHRx), or tertiary (—NRxRy) group, wherein Rx and Ry is any alkyl, aryl, heterocyclyl, cycloalkyl or alkenylene, each optionally and independently substituted with one or more substituents described herein. The Rx and Ry substituents may be taken together to form a “ring,” wherein the “ring,” as used herein, is cyclic amino groups such as piperidine and pyrrolidine, and may include heteroatoms such as in morpholine, and may be optionally substituted in the same manner as alkyl, described above. The terms “alkylamino,” “alkenylamino,” or “alkynylamino” as used herein, refer to an alkyl group, an alkenyl group, or an alkynyl group, as defined herein, substituted with an amino group.

The term “alkenyl,” as used herein, refers to a straight-chain or branched alkyl group having one or more carbon-carbon double bonds. Thus, “(C2-C6) alkenyl”, for example, means a radical having 2-6 carbon atoms in a linear or branched arrangement having one or more double bonds. Examples of alkenyl groups include, but are not limited to, ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene).

The term “alkynyl,” as used herein, refers to a straight-chain or branched alkyl group having one or more carbon-carbon triple bonds. Thus, “(C2-C6) alkynyl”, for example, means a radical having 2-6 carbon atoms in a linear or branched arrangement having one or more triple bonds. Examples of alkynyl groups include, but are not limited to, ethynyl, propynyl, butynyl, pentynyl, and the like. The one or more carbon-carbon triple bonds can be internal (such as in 2-butyne) or terminal (such as in 1-butyne).

The term “alkoxy”, as used herein, refers to an “alkyl-O—” group, wherein alkyl is defined above. Examples of alkoxy group include methoxy or ethoxy groups.

The terms “halogen” or “halo,” as used herein, refer to fluorine, chlorine, bromine or iodine.

The term “aryl,” as used herein, refers to an aromatic monocyclic or polycyclic (e.g. bicyclic or tricyclic) carbocyclic ring system. Thus, “(C6-C18) aryl”, for example, is a 6-18 membered monocylic or polycyclic system. Aryl systems include optionally substituted groups such as phenyl, biphenyl, naphthyl, phenanthryl, anthracenyl, pyrenyl, fluoranthyl or fluorenyl. An aryl can be optionally substituted. Examples of suitable substituents on an aryl include halogen, hydroxyl, (C1-C12) alkyl, (C2-C6) alkenyl, (C2-C6) alkynyl, (C1-C6) haloalkyl, (C1-C3) alkylamino, (C1-C3) dialkylamino (C1-C6) alkoxy, (C6-C18) aryloxy, (C6-C18) arylamino, (C6-C18) aryl, (C6-C18) haloaryl, (5-12 atom) heteroaryl, —NO2, —CN, —OF3 and oxo.

In some embodiments, a (C6-C18) aryl is phenyl, indenyl, naphthyl, azulenyl, heptalenyl, biphenyl, indacenyl, acenaphthylenyl, fluorenyl, phenalenyl, phenanthrenyl, anthracenyl, cyclopentacyclooctenyl or benzocyclooctenyl. In some embodiments, a (C6-C18) aryl is phenyl, naphthalene, anthracene, 1H-phenalene, tetracene, and pentacene.

The term “heteroaryl,” as used herein, refers aromatic groups containing one or more atoms is a heteroatom (0, S or N). A heteroaryl group can be monocyclic or polycyclic, e.g., a monocyclic heteroaryl ring fused to one or more carbocyclic aromatic groups or other monocyclic heteroaryl groups. The heteroaryl groups of this invention can also include ring systems substituted with one or more oxo moieties. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

In other embodiments, a 5-20-membered heteroaryl group is pyridyl, 1-oxo-pyridyl, furanyl, benzo[1,3]dioxolyl, benzo[1,4]dioxinyl, thienyl, pyrrolyl, oxazolyl, imidazolyl, thiazolyl, a isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, benzothienyl.

The term “haloalkyl,” as used herein, includes an alkyl substituted with one or more F, Cl, Br, or I, wherein alkyl is defined above.

The term “haloaryl,” as used herein, includes an aryl substituted with one or more F, Cl, Br, or I, wherein aryl is defined above.

The term “hetero,” as used herein, refers to the replacement of at least one carbon atom member in a ring system with at least one heteroatom selected from N, S or O. “Hetero” also refers to the replacement of at least one carbon atom member in an acyclic system. A hetero ring system or a hetero acyclic system may have 1, 2, or 3 carbon atom members replaced by a heteroatom.

The terms “heterocycle” or “heterocyclyl” or “heterocyclic,” as used herein, refer to a saturated or unsaturated group having a single ring or multiple condensed rings, from 1 to 10 carbon atoms and from 1 to 4 heteroatoms selected from nitrogen, sulfur or oxygen. In fused ring systems, one or more of the rings can be aryl or heteroaryl, provided that the point of attachment is at the heterocyclyl. Heterocyclyl can be unsubstituted or substituted in accordance with cycloalkyl.

The term “oxo,” as used herein, refers to ═O. When an oxo group is a substituent on a carbon atom, they form a carbonyl group (C(O)).

The term “nitro,” as used herein, refers to —NO₂.

The term “nitrile,” as used herein, refers to —C≡N.

The term “pyridyl,” as used herein, refers to —C₅H₄N, wherein the location of the nitrogen atom in the ring may vary.

The term “4-5 member polycyclyl” is a cyclic compound with 4-5 hydrocarbon loop or ring structures (e.g., benzene rings). The term generally includes all polycyclic aromatic compounds, including the polycyclic aromatic hydrocarbons, the heterocyclic aromatic compounds containing sulfur, nitrogen, oxygen, or another non-carbon atoms, and substituted derivatives of these. A polycyclyl can be fused to another ring to create a fused bicyclic or polycyclic system. An example of a ring substituted with a 4-5 member polycyclyl includes, for example:

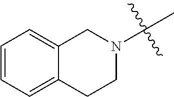

wherein [symbol] represents a point of attachment between two atoms.

The term “target cell,” as used herein, refers to any cell in which visualization is desired. An example of a target cell is neural stem cell. In an example embodiment, the neural stem cell has a low level of Abcg2.

### REFERENCES

1. Vendrell M, Lee J S, & Chang Y T (2010) Diversity-oriented fluorescence library approaches for probe discovery and development. Curr Opin Chem Biol 14(3):383-389.
2. Yun S W, et al. (2014) Diversity oriented fluorescence library approach (DOFLA) for live cell imaging probe development. Acc Chem Res 47(4):1277-1286.
3. Mouthon M A, et al. (2006) Neural stem cells from mouse forebrain are contained in a population distinct from the ‘side population’. J Neurochem 99(3):807-817.
4. Orford M, et al. (2009) Generation of an ABCG2(GFPn-puro) transgenic line—a tool to study ABCG2 expression in mice. Biochem Biophys Res Commun 384(2):199-203.
5. Szakacs G, et al. (2004) Predicting drug sensitivity and resistance: profiling ABC transporter genes in cancer cells. Cancer Cell 6(2):129-137.
6. Robey R W, et al. (2004) Pheophorbide a is a specific probe for ABCG2 function and inhibition. Cancer Res 64, 1242-1246.
7. Yun S W, et al. (2012) Neural stem cell specific fluorescent chemical probe binding to FABP7. Proc Natl Acad Sci USA 109(26):10214-10217.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A composition represented by structural formula (I): 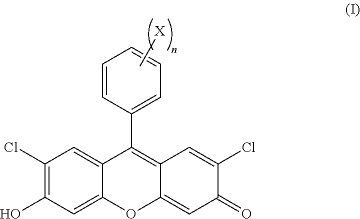
or a salt and/or tautomer thereof, wherein
n is a whole number selected from 1 to 5;
X for each occurrence is independently selected from H, (C₁-C₂₀)alkyl, (C₂-C₂₀)alkenyl, (C₂-C₂₀)alkynyl, (C₁-C₂₀)alkoxy, (C₁-C₂₀)alkylamino, (C₃-C₁₀)cycloalkyl, —C(O)R₁, —S(O)₂R₁, amino, pyridyl, nitrile, nitro or —C(O)N(R₁)(R₂);
R₁ is H, amino, (C₁-C₂₀)alkyl, (C₂-C₂₀)alkenyl, (C₂-C₂₀)alkynyl, (C₁-C₂₀)alkoxy, (C₁-C₂₀)alkylamino or (C₃-C₁₀)cycloalkyl, optionally substituted with one or more groups independently selected from (C₁-C₁₀)alkyl, (C₃-C₁₀)cycloalkyl, halo, (C₆-C₁₂)aryl, (5-12 atom) heteroaryl, (5-12 atom) heterocycle, or —C(O)O(C₁-C₃)alkyl, further optionally substituted with one or more groups selected from halo, (C₆-C₁₂)aryl, (C₁-C₃)alkyl, (C₁-C₃)alkoxy, —OCF₃ or oxo;
R₂ is H, amino, (C₁-C₂₀)alkyl, (C₂-C₂₀)alkenyl, (C₂-C₂₀)alkynyl, (C₁-C₂₀)alkoxy, (C₁-C₂₀)alkylamino or (C₃-C₁₀)cycloalkyl, optionally substituted with one or more groups independently selected from (C₁-C₁₀)alkyl, (C₃-C₁₀)cycloalkyl, halo, (C₆-C₁₂)aryl, (5-12 atom) heteroaryl, (5-12 atom) heterocycle, or —C(O)O(C₁-C₃)alkyl, further optionally substituted with one or more groups selected from halo, (C₆-C₁₂)aryl, (C₁-C₃)alkyl, (C₁-C₃)alkoxy, —OCF₃ or oxo;
or R₁ and R₂ may be taken together to form a ring, wherein the ring is optionally substituted with one or more groups selected from (C₁-C₁₀)alkyl, (C₃-C₁₀)cycloalkyl, halo, (C₆-C₁₂)aryl, (5-12 atom) heteroaryl, (5-12 atom) heterocycle, —C(O)O(C₁-C₃)alkyl, or a 4-5 member polycyclyl fused to the ring, further optionally substituted with one or more groups selected from halo, (C₆-C₁₂)aryl, (C₁-C₃)alkyl, (C₁-C₃)alkoxy, —OCF₃, or oxo;
with the proviso that when the composition of structural formula I is represented by structural formula (II): 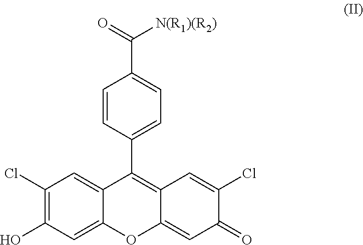
R₁ and R₂ cannot both be n-hexyl.

2. The composition of claim 1, wherein X is —C(O)R₁, —S(O)₂R₁ or —C(O)N(R₁)(R₂).

3. The composition of claim 1, wherein X is —C(O)N(R₁)(R₂).

4. The composition of claim 1, wherein X is —C(O)N(R₁)(R₂), and R₁ and R₂ are independently (C₅-C₁₂)alkyl.

5. The composition of claim 1, wherein X is —C(O)N(R₁)(R₂), and R₁ and R₂ are independently (C₆-C₉)alkyl.

6. The composition of claim 1, wherein X is —C(O)N(R₁)(R₂) at para position, and R₁ and R₂ are independently (C₆-C₉)alkyl.

7. The composition of claim 1, wherein formula (I) is represented by the structural formula:

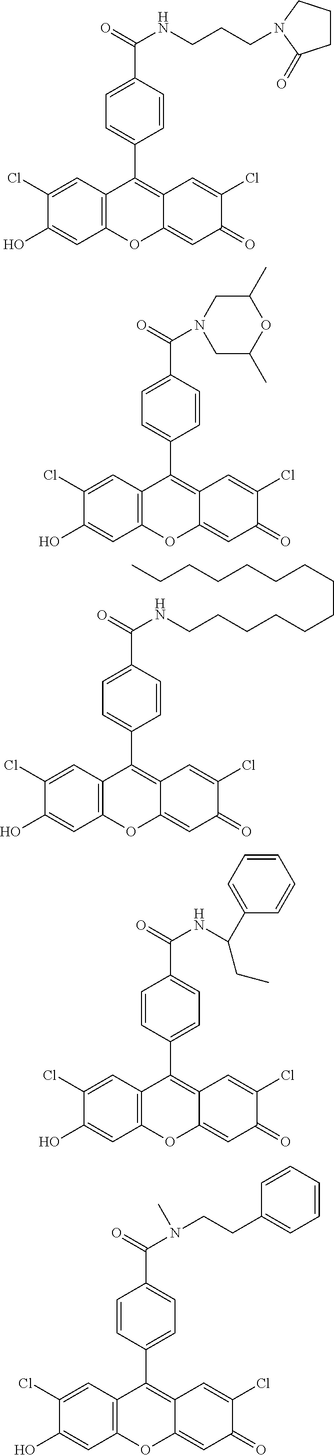

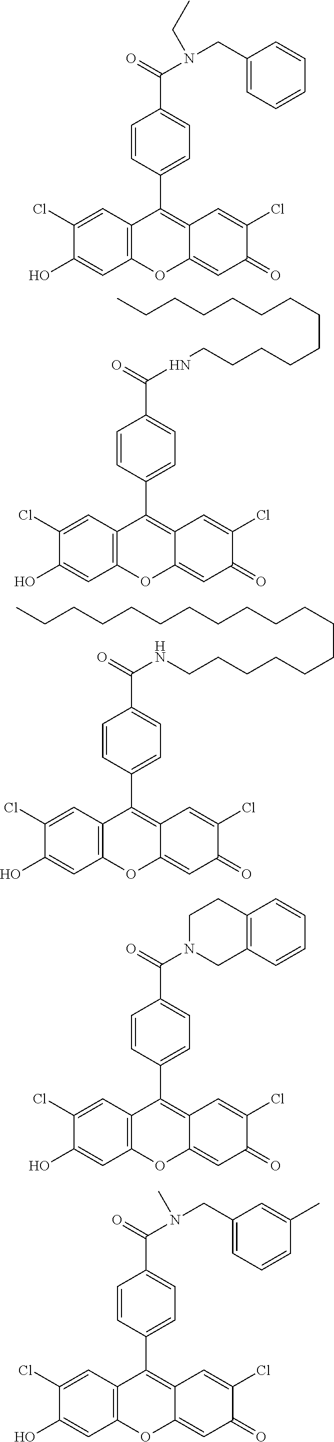

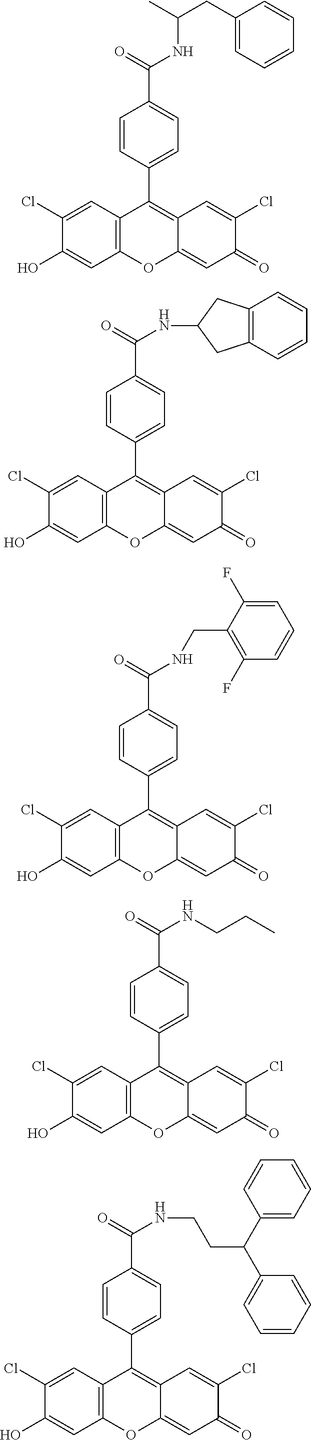

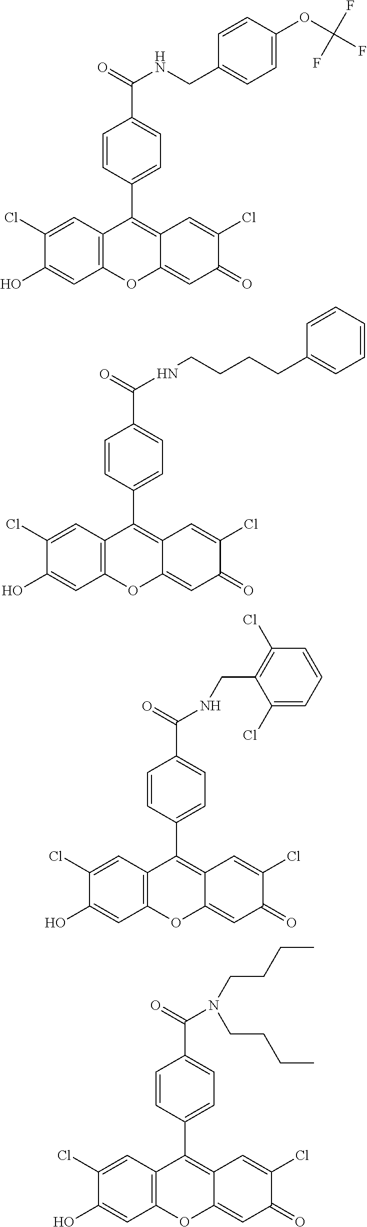

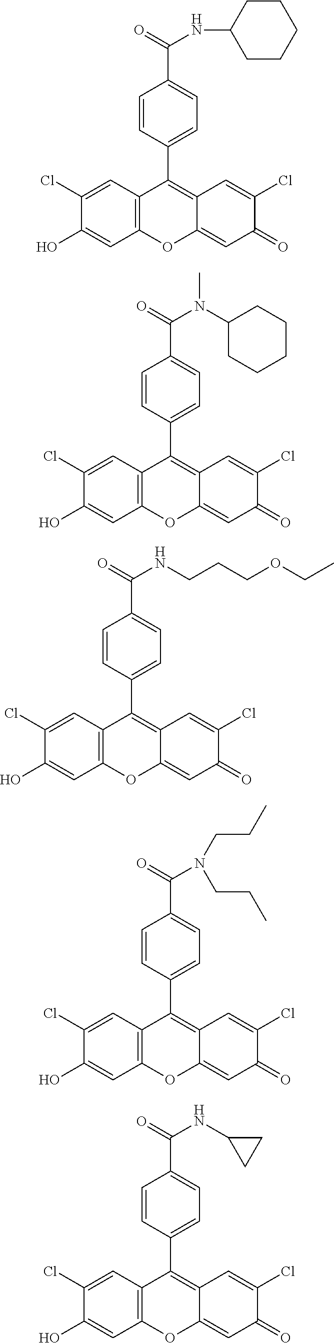

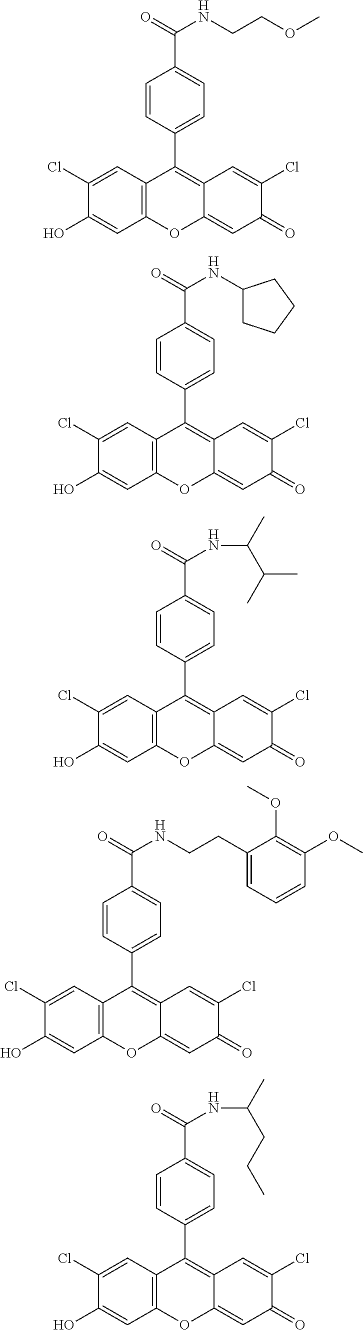

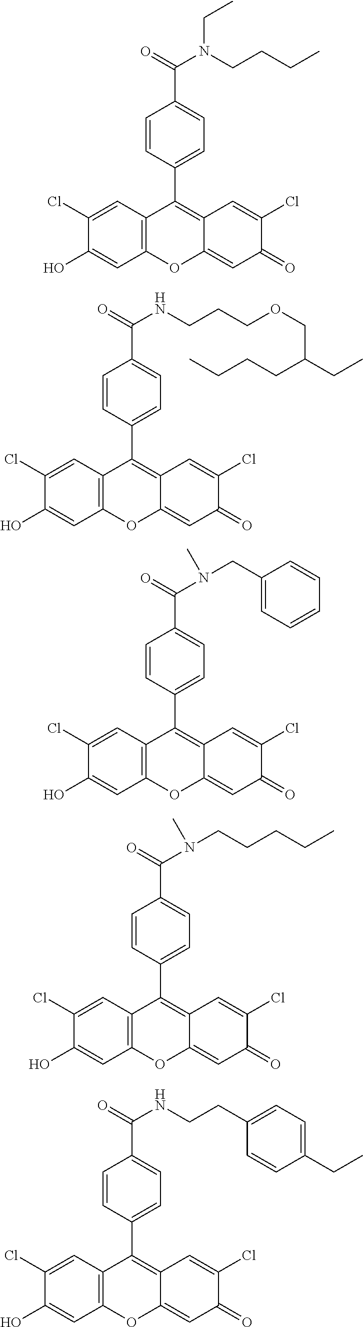

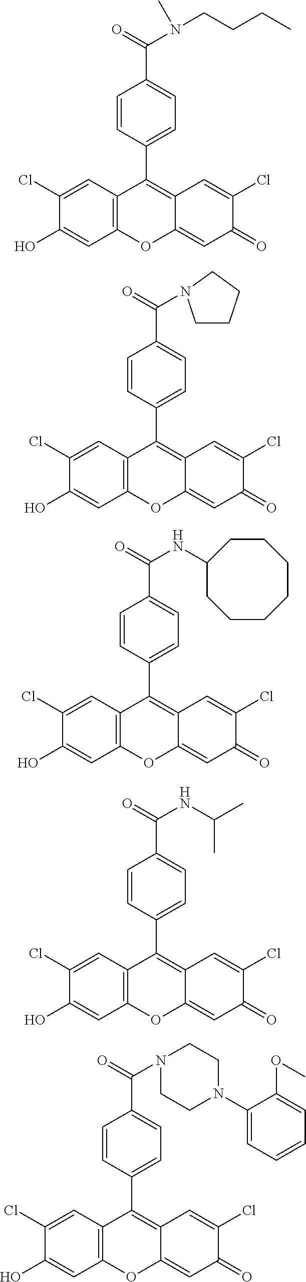

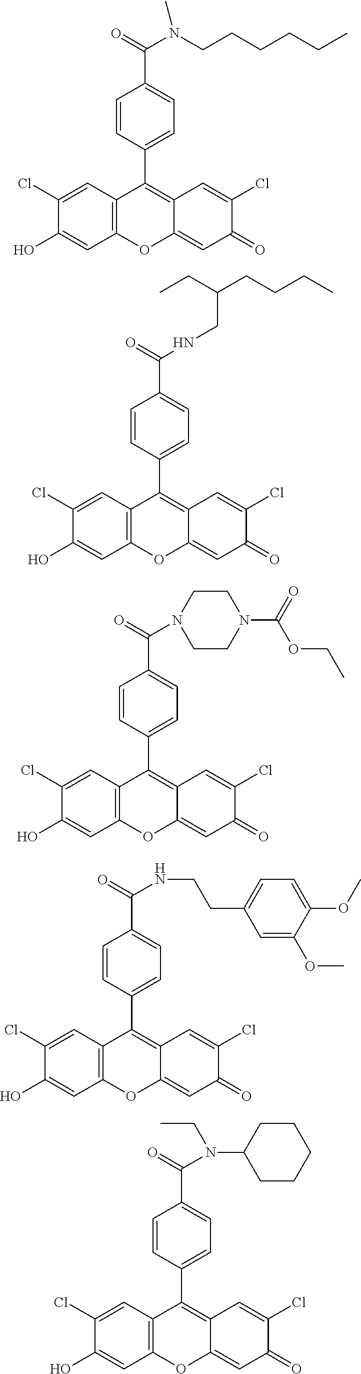

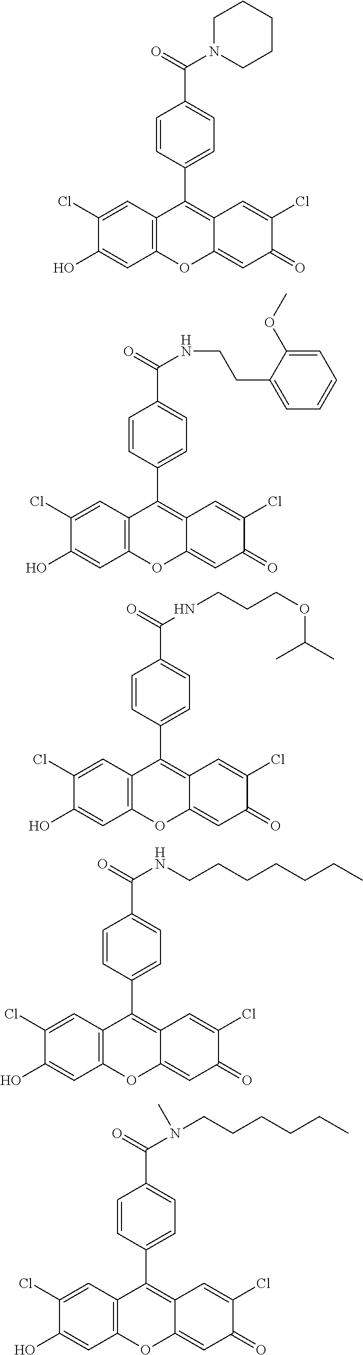

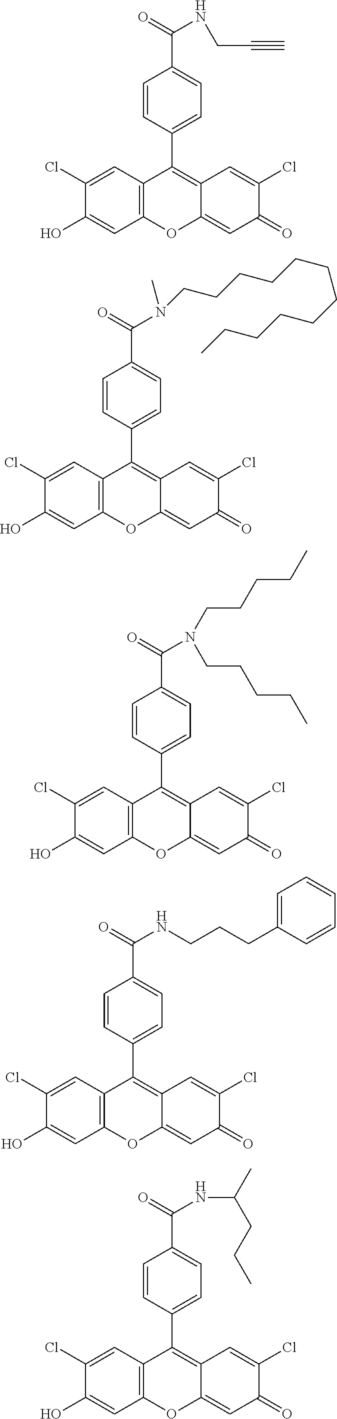

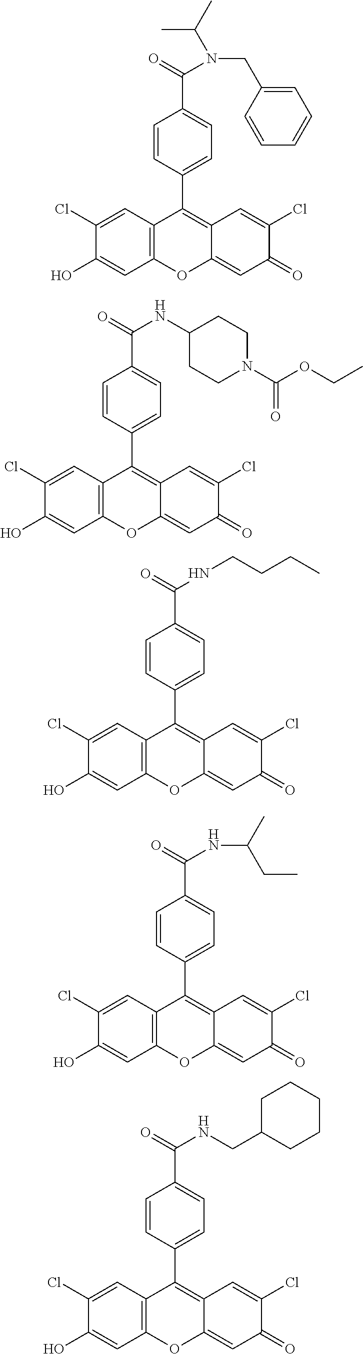

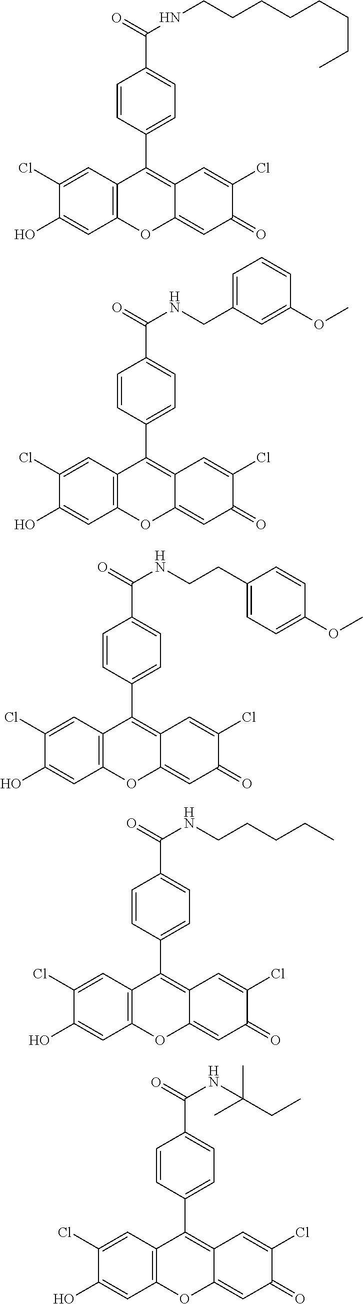

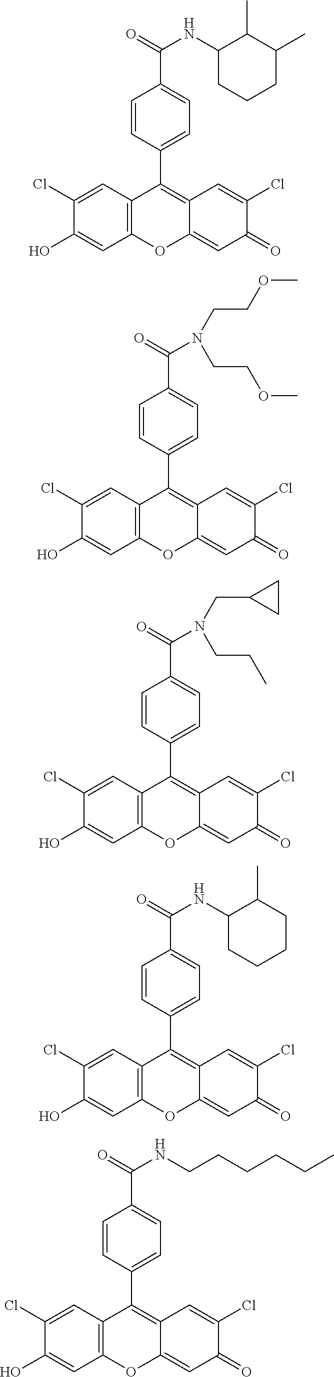

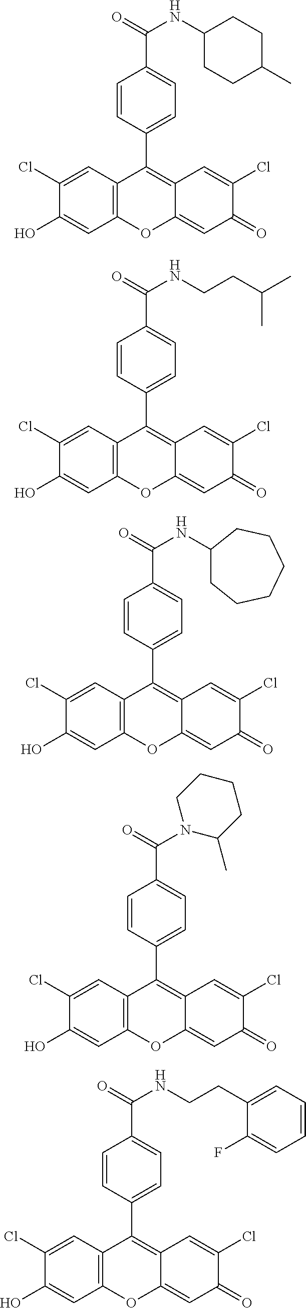

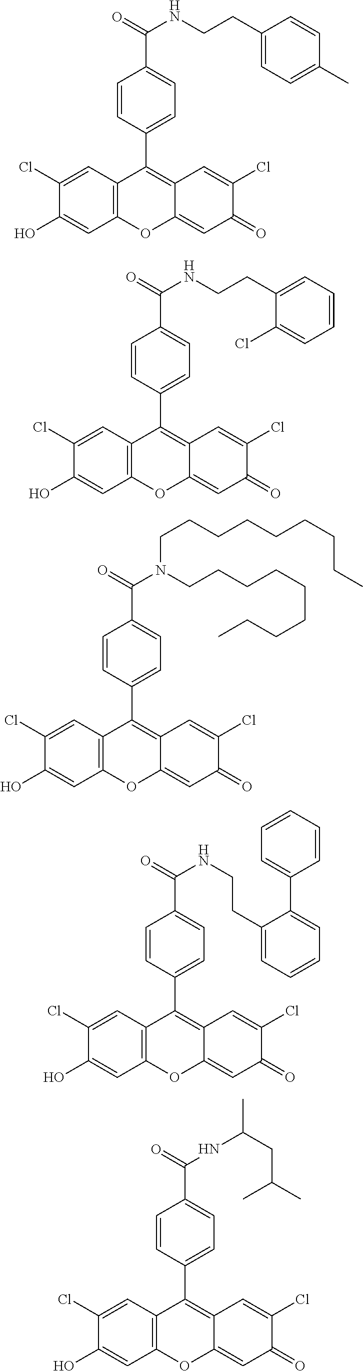

8. A method of visualizing a target cell, the method comprising:
a) contacting a population of the target cell with a composition to form an incubation media;
b) incubating the incubation media of step (a) for a period of time sufficient to stain the target cells; and
c) visualizing the stained target cells of step (b) with fluorescence microscopy to visualize the target cell;
wherein the composition is represented by structural formula (I): 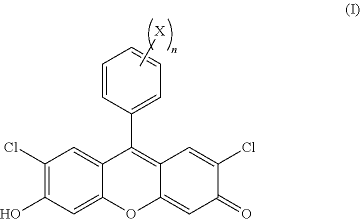
or a salt and/or a tautomer thereof, wherein
n is a whole number selected from 1 to 5;
X for each occurrence is independently selected from H, (C₁-C₂₀)alkyl, (C₂-C₂₀)alkenyl, (C₂-C₂₀)alkynyl, (C₁-C₂₀)alkoxy, (C₁-C₂₀)alkylamino, (C₃-C₁₀)cycloalkyl, —C(O)R₁, —S(O)₂R₁, amino, pyridyl, nitrile, nitro or —C(O)N(R₁)(R₂);
R₁ is H, amino, (C₁-C₂₀)alkyl, (C₂-C₂₀)alkenyl, (C₂-C₂₀)alkynyl, (C₁-C₂₀)alkoxy, (C₁-C₂₀)alkylamino or (C₃-C₁₀)cycloalkyl, optionally substituted with one or more groups independently selected from (C₁-C₁₀)alkyl, (C₃-C₁₀)cycloalkyl, halo, (C₆-C₁₂)aryl, (5-12 atom) heteroaryl, (5-12 atom) heterocycle, or —C(O)O(C₁-C₃)alkyl, further optionally substituted with one or more groups selected from halo, (C₆-C₁₂)aryl, (C₁-C₃)alkyl, (C₁-C₃)alkoxy, —OCF₃ or oxo;
R₂ is H, amino, (C₁-C₂₀)alkyl, (C₂-C₂₀)alkenyl, (C₂-C₂₀)alkynyl, (C₁-C₂₀)alkoxy, (C₁-C₂₀)alkylamino or (C₃-C₁₀)cycloalkyl, optionally substituted with one or more groups independently selected from (C₁-C₁₀)alkyl, (C₃-C₁₀)cycloalkyl, halo, (C₆-C₁₂)aryl, (5-12 atom) heteroaryl, (5-12 atom) heterocycle, or —C(O)O(C₁-C₃)alkyl, further optionally substituted with one or more groups selected from halo, (C₆-C₁₂)aryl, (C₁-C₃)alkyl, (C₁-C₃)alkoxy, —OCF₃ or oxo;
or R₁ and R₂ may be taken together to form a ring, wherein the ring is optionally substituted with one or more groups selected from (C₁-C₁₀)alkyl, (C₃-C₁₀)cycloalkyl, halo, (C₆-C₁₂)aryl, (5-12 atom) heteroaryl, (5-12 atom) heterocycle, —C(O)O(C₁-C₃)alkyl, or a 4-5 member polycyclyl fused to the ring, further optionally substituted with one or more groups selected from halo, (C₆-C₁₂)aryl, (C₁-C₃)alkyl, (C₁-C₃)alkoxy, —OCF₃, or oxo.

9. The method of claim 8, wherein the target cell is a neural stem cell.

10. The method of claim 9, wherein the neural stem cell is an ABCG2^{low} neural stem cell.

11. The method of claim 8, wherein X is —C(O)R₁, —S(O)₂R₁ or —C(O)N(R₁)(R₂).

12. The method of claim 8, wherein X is —C(O)N(R₁)(R₂).

13. The method of claim 8, wherein X is —C(O)N(R₁)(R₂), and R₁ and R₂ are independently (C₅-C₁₂)alkyl.

14. The method of claim 8, wherein X is —C(O)N(R₁)(R₂), and R₁ and R₂ are independently (C₆-C₉)alkyl.

15. The method of claim 8, wherein X is —C(O)N(R₁)(R₂) at para position, and R₁ and R₂ are independently (C₆-C₉)alkyl.

16. A method of isolating a neural stem cell, the method comprising:
a) visualizing the neural stem cell by contacting a population of the neural stem cells with a composition to form an incubation media, incubating the incubation media for a period of time sufficient to stain the neural stem cells, and visualizing the stained neural stem cells with fluorescence microscopy to visualize the neural stem cell;
b) exciting the neural stem cells by exposing the incubation media to light of a wavelength of about 488 nm to about 561 nm; and
c) separating the excited neural stem cells from the incubation media by fluorescence activated cell sorting using a bandpass filter configured to detect light emitted at about 529±28 nm;
wherein the composition is represented by structural formula (I): 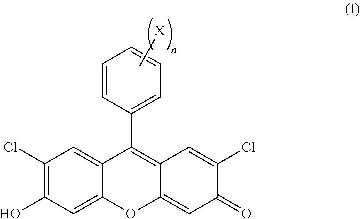
or a salt and/or tautomer thereof, wherein
n is a whole number selected from 1 to 5;
X for each occurrence is independently selected from H, (C₁-C₂₀)alkyl, (C₂-C₂₀)alkenyl, (C₂-C₂₀)alkynyl, (C₁-C₂₀)alkoxy, (C₁-C₂₀)alkylamino, (C₃-C₁₀)cycloalkyl, —C(O)R₁, —S(O)₂R₁, amino, pyridyl, nitrile, nitro or —C(O)N(R₁)(R₂);
R₁ is H, amino, (C₁-C₂₀)alkyl, (C₂-C₂₀)alkenyl, (C₂-C₂₀)alkynyl, (C₁-C₂₀)alkoxy, (C₁-C₂₀)alkylamino or (C₃-C₁₀)cycloalkyl, optionally substituted with one or more groups independently selected from (C₁-C₁₀)alkyl, (C₃-C₁₀)cycloalkyl, halo, (C₆-C₁₂)aryl, (5-12 atom) heteroaryl, (5-12 atom) heterocycle, or —C(O)O(C₁-C₃)alkyl, further optionally substituted with one or more groups selected from halo, (C₆-C₁₂)aryl, (C₁-C₃)alkyl, (C₁-C₃)alkoxy, —OCF₃ or oxo;
R₂ is H, amino, (C₁-C₂₀)alkyl, (C₂-C₂₀)alkenyl, (C₂-C₂₀)alkynyl, (C₁-C₂₀)alkoxy, (C₁-C₂₀)alkylamino or (C₃-C₁₀)cycloalkyl, optionally substituted with one or more groups independently selected from (C₁-C₁₀)alkyl, (C₃-C₁₀)cycloalkyl, halo, (C₆-C₁₂)aryl, (5-12 atom) heteroaryl, (5-12 atom) heterocycle, or —C(O)O(C₁-C₃)alkyl, further optionally substituted with one or more groups selected from halo, (C₆-C₁₂)aryl, (C₁-C₃)alkyl, (C₁-C₃)alkoxy, —OCF₃ or oxo;
or R₁ and R₂ may be taken together to form a ring, wherein the ring is optionally substituted with one or more groups selected from (C₁-C₁₀)alkyl, (C₃-C₁₀)cycloalkyl, halo, (C₆-C₁₂)aryl, (5-12 atom) heteroaryl, (5-12 atom) heterocycle, —C(O)O(C₁-C₃)alkyl, or a 4-5 member polycyclyl fused to the ring, further optionally substituted with one or more groups selected from halo, (C₆-C₁₂)aryl, (C₁-C₃)alkyl, (C₁-C₃)alkoxy, —OCF₃, or oxo.

17. The method of claim 16, wherein X is —C(O)R₁, —S(O)₂R₁ or —C(O)N(R₁)(R₂).

18. The method of claim 16, wherein X is —C(O)N(R₁)(R₂).

19. The method of claim 16, wherein X is —C(O)N(R₁)(R₂), and R₁ and R₂ are independently (C₅-C₁₂)alkyl.

20. The method of claim 16, wherein X is —C(O)N(R₁)(R₂), and R₁ and R₂ are independently (C₆-C₉)alkyl.

21. The method of claim 16, wherein X is —C(O)N(R₁)(R₂) at para position, and R₁ and R₂ are independently (C₆-C₉)alkyl.
